(19) United States
(12) Patent Application Publication
(10) Pub. No.: US 2024/0043745 A1
(43) Pub. Date: Feb. 8, 2024

(54) USE OF EUGENOL DERIVATIVES AS STABILIZERS, ORGANIC MATERIAL AND EUGENOL DERIVATIVES

(71) Applicant: FRAUNHOFER-GESELLSCHAFT ZUR FÖRDERUNG DER ANGEWANDTEN FORSCHUNG E.V., München (DE)

(72) Inventors: Jannik MAYER, Darmstadt (DE); Elke METZSCH-ZILLIGEN, Darmstadt (DE); Rudolf PFAENDNER, Darmstadt (DE)

(73) Assignee: FRAUNHOFER-GESELLSCHAFT ZUR FÖRDERUNG DER ANGEWANDTEN FORSCHUNG E.V., München (DE)

(21) Appl. No.: 18/251,255

(22) PCT Filed: Nov. 2, 2021
(86) PCT No.: PCT/EP2021/080340

§ 371 Date: May 1, 2023

(30) Foreign Application Priority Data
Nov. 2, 2020 (DE) 10 2020 128 803.4

(51) Int. Cl. C09K 15/14; C07C 323/52; C07D 493/04; C08F 20/38; C08L 23/12; C08K 5/375; C08K 5/378

(52) U.S. Cl. CPC C09K 15/14; C07C 323/52; C07D 493/04; C08F 20/38; C08L 23/12; C08K 5/375; C08K 5/378; C08L 2201/08

(57) ABSTRACT

The present invention relates to the use of specific eugenol derivatives as stabilizers of organic materials against oxidative, thermal and/or actinic degradation. The present invention also relates to stabilized organic material and to specific eugenol derivatives which are suitable as stabilizers.

The present invention relates to the use of specific eugenol derivatives as stabilizers of organic materials against oxidative, thermal and/or actinic degradation. In addition, the present invention relates to a stabilized organic material and specific eugenol derivatives that are suitable as stabilizers.

Organic materials such as plastics are subject to aging processes that ultimately result in a loss of the desired properties such as of the mechanical characteristic values. This process, called autoxidation, leads to changes in the polymer chain, for example, in molecular weight or the formation of new chemical groups, arising from radical chain cleavages through mechanochemical processes or through UV radiation in the presence of oxygen. Stabilizers are therefore used to prevent or at least delay said aging. Important representatives of stabilizers are antioxidants, which interfere with the free radicals formed during autoxidation and thus interrupt the degradation process. A distinction is generally made between primary antioxidants, which can react directly with oxygen-comprising free radicals or C-radicals, and secondary antioxidants, which react with intermediately formed hydroperoxides (see C. Kröhnke et al. Antioxidants in Ullmann's encyclopedia of industrial chemistry, Wiley-VCH Verlag, Weinheim 2015). Typical representatives of primary antioxidants are, for example, phenolic antioxidants, amines, but also lactones. Classes of secondary antioxidants are phosphorus compounds such as phosphites and phosphonites, but also organosulfur compounds such as thioesters, thioethers and disulfides. Primary and secondary antioxidants are typically frequently combined in practice, which produces a synergistic effect. It is also possible and known to combine primary and secondary antioxidant groups in one molecule, for example a phenolic antioxidant group and a phosphite, such as described in EP 823435 (Sumitomo Chemical Company) or the combination of a phenolic antioxidant group with sulfur compounds, as is known from EP224442 (Ciba-Geigy AG), from U.S. Pat. No. 3,334,046 (Geigy Chemical Corporation), from U.S. Pat. No. 42,282,971 (Ciba-Geigy AG) and from WO 2019/096868 (Fraunhofer Gesellschaft).

Plastics formed from fossil raw materials such as petroleum or natural gas are increasingly being supplemented or replaced by plastics based on renewable raw materials obtained via biochemical processes. The question of sustainability then also arises for the primary and secondary antioxidants used therefor (and for plastics made from fossil raw materials). There is therefore a need for stabilizers based on renewable and available raw materials that are highly effective, have low volatility and are compatible with polymeric substrates. A high efficacy can be achieved, as already mentioned, through the combination of primary and secondary antioxidant functions, among other possibilities. It was therefore the object of the present invention to provide stabilizers, which have primary and secondary antioxidant structures in one molecule and can be obtained at least partially from easily accessible renewable raw materials.

Antioxidants made from renewable raw materials, which are also occasionally used in plastics, are basically known. Tocopherols (vitamin E) are a typical example. Tocopherols like customary antioxidants have a sterically hindered phenol structure and can be used alone or in combination with secondary antioxidants (e.g. S. Al-Malaika, Macromol. Symp. 2001, 176, 107-117). Tocopherols can e.g. be isolated from natural products such as wheat germ oil or olive oil. Further known phenolic antioxidants made of natural substances which were examined in plastics are, for example,
quercetin (B. Kirschweng et al., Melt stabilisation of PE with natural antioxidants: Comparison of rutin and quercetin, Eur. Pol. J. 2018, 103, 228-237,
dihydromyrecitin (B. Kirschweng et al., Melt stabilization of polyethylene with dihydromyrecitin, a natural antioxidant, Pol. Degr. Stab. 2016, 133, 192-200,
derivatives of rosmarinic acid (K. Doudin et al., New genre of antioxidants from renewable natural resources: Synthesis and characterization of rosemary plant-derived antioxidants and their performance in polyolefins, Pol. Degr. Stab. 2016, 130, 126-134,
tannins (W. J. Grigsby et al., Esterification of condensed tannins and their impact on the properties of poly (lactic acid), Polymers 5 (2013) 344-360),
curcumin (D. Tatraaljai et al. Processing stabilisation of PE with a natural antioxidant, curcumin, European Polymer Journal 49 (2013) 1196-1203, and
silymarin (B. Kirschweng et al., Melt stabilization of polyethylene with natural antioxidants: comparison of a natural extract and its main components. Journal of Thermal Analysis and Calorimetry https://doi.org/10.1007/s10973-020-09709-5.
catechins from tea and coffee extracts (O. Olejnik, A. Masek, Bio-Based Packaging Materials Containing Substances Derived from Coffee and Tea Plants, Materials 2020, 13, 5719)

What all of these stabilizers made from renewable raw materials have in common is that they occur in relatively low concentrations in the respective starting materials, mostly plant-based raw materials, which makes isolation, purification or the production of usable secondary products disproportionately expensive. In contrast, eugenol is a raw material which occurs, on the one hand, in high concentrations in many plants (see A. A. Khalil et al., Essential oil eugenol: sources, extraction techniques and nutraceutical perspectives RSC Adv., 2017, 7, 32669) but can also be produced from lignin, which is available in large quantities (e.g. CN 105669397). Eugenol is therefore an attractive compound with potential antioxidant effect; however, it cannot be used as such in the usual processing methods for plastics due to the high volatility.

The reaction of eugenol with thiols via click reaction is known (M. Shrestha et al., Aliphatic-Aromatic Polyols by Thiol-Ene Reactions, Journal of Polymers and the Environment (2018), 26(6), 2257-2267). In our own application WO 2019/096868, click reactions for the production of sulfur-containing antioxidants were likewise claimed; however, the compounds described there necessarily have a steric hindrance and are therefore to be distinguished structurally from the present structures in accordance with the invention. The compounds in accordance with the invention are also not included therein. Furthermore, eugenol sulfur compounds are used as components for producing coatings (H. Watanabe et al. Biobased Coatings Based on Eugenol Derivatives, ACS Applied Bio Materials (2018), 1(3), 808-813, Y. Hu et al. Synthesis of Eugenol-Based Polyols via Thiol-Ene Click Reaction and High-Performance Thermosetting Polyurethane Therefrom, ACS Sustainable Chemistry & Engineering (2020), 8(10), 4158-4166). Further reactive components based on eugenol-sulfur derivatives have been described as intermediate products, e.g. for self-healing polymers in the form of epoxides (C. Cheng et al. J. Pol. Res. (2018), 25, 1-13).

The solution to the previously mentioned problem is provided in the independent patent claims, wherein patent claim 1 relates to the use of a specific eugenol derivative as a stabilizer, patent claim 9 relates to a stabilized organic material and patent claim 13 relates to specific eugenol derivatives.

The present object of producing effective antioxidants for plastics from readily available renewable raw materials could then be solved by proposing new sulfur-containing stabilizers using eugenol as the raw material. Surprisingly, these stabilizers show a particularly good effect when stabilizing plastics and an excellent thermal stability.

The present invention thus relates to the use of a compound in accordance with the general Formula I

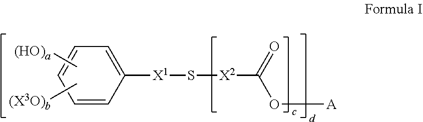

wherein, respectively independent of one another,
X¹ is a linear or branched alkylene radical having 2 to 18 carbon atoms,
X² is a linear or branched alkylene radical having 1 to 18 carbon atoms,
X³ is a linear or branched alkyl radical having 1 to 18 carbon atoms,
A is a d-valent saturated or unsaturated group,
a 2 to 5-b,
b 0 to 3,
c 0 or 1, and
d 1 to 8,
or
a polymeric compound containing a repeating unit according to general Formula II

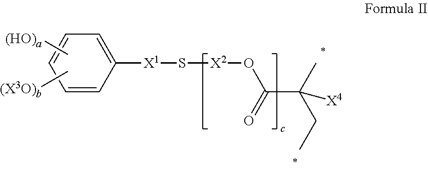

wherein
X¹, X², X³, a, b and c are defined as previously, and
X⁴ is hydrogen or a linear or branched alkyl radical having 1 to 18 carbon atoms, and
* is an attachment site of the repeating unit according to general Formula II,
or a mixture of a plurality of the compounds according to general Formula I and/or polymeric compounds containing a repeating unit according to general Formula II,
as a stabilizer of organic materials, in particular plastics, against oxidative, thermal and/or actinic degradation.

Thus, new stabilizers and stabilizer compositions based on easily available renewable raw materials and a new method for stabilizing plastics are proposed, which have a good efficacy, a high thermal stability, are environmentally friendly and have a favorable cost structure.

Stabilizers are commercially-available products which, depending on the combination of properties, cover various market segments for plastics, coatings and oils/fats. The new stabilizers and stabilizer combinations are previously unknown substances made at least partially from renewable raw materials.

The structures of the present stabilizers in accordance with the invention have previously not been used for stabilizing plastics, the structures have occasionally also not been previously described in the literature.

In one preferred embodiment, the present invention provides the use according to claim 1, characterized in that
X¹ is a linear alkylene radical having 2 to 6, preferably 3 carbon atoms,
X² is a linear alkylene radical having 1 to 6, preferably 1 to 4, particularly preferably 1 or 2 carbon atoms,
X³ is a linear or branched alkyl radical having 1 to 4 carbon atoms,
X⁴ is hydrogen or a linear or branched alkyl radical having 1 to 4 carbon atoms, in particular methyl,
A is a d-valent, aliphatic or aromatic, preferably linear or branched, aliphatic group having 1 to 32, preferably 1 to 24, particularly preferably 1 to 18 carbon atoms,
a 2 and
b 0.

In particular, the above mentioned compounds of Formula I, the polymeric compounds containing a repeating unit according to general Formula II or the mixtures of a plurality of the compounds according to general Formula I and/or polymeric compounds containing a repeating unit according to general Formula II, are suitable as stabilizers for plastics in the form of injection molded parts, foils or films, foams, fibers, cables and pipes, profiles, hollow bodies, ribbons, membranes, such as e.g. geomembranes, or adhesives produced via extrusion, injection molding, blow molding, calendering, pressing processes, spinning processes, rotomolding, for example, for the electrical and electronics industry, construction industry, transport industry (car, aircraft, ship, train), for medical applications, for household and electrical appliances, vehicle parts, consumer goods, packaging, furniture, textiles. A further area of application are paints, colorants and coatings, and oils and fats.

If the organic materials are oils and fats, they can be based on mineral oils, vegetable fats, or animal fats, or also oils, fats, or waxes based on e.g. synthetic esters. Vegetable oils and fats are, for example, palm oil, olive oil, rapeseed oil, linseed oil, soybean oil, sunflower oil, castor oil; animal fats are, for example, fish oils or beef tallow. The compounds in accordance with the invention can furthermore be used as stabilizers of lubricants, hydraulic oils, engine oils, turbine oils, transmission oils, metal machining fluids, or as lubricating greases. These mineral or synthetic lubricants are primarily based on hydrocarbons. The chemical products are for example for stabilizing polyols in polyurethane production.

The compounds of Formula I in accordance with the invention can furthermore be used as stabilizers of lubricants, hydraulic oils, engine oils, turbine oils, transmission oils, metal machining fluids, or as lubricating greases. These mineral or synthetic lubricants are primarily based on hydrocarbons.

The incorporation of the compounds of the formula described above, the polymeric compounds, containing a repeating unit according to general Formula II or the mixture of a plurality of the compounds according to general Formula I and/or polymeric compounds containing a repeating unit according to general Formula II, I and, optionally, additional additives into the organic material, e.g., the plastic, is carried out by usual processing methods, wherein, for example, the polymer is melted and mixed with the additive composition in accordance with the invention and the optional further additives, preferably by mixers, kneaders or extruders. Extruders such as single screw extruders, twin screw extruders, planetary gear extruders, ring extruders, and co-kneaders that are preferably equipped with a vacuum degassing are preferred as processing machines. The processing can take place under air or optionally under inert gas conditions such as under nitrogen.

Furthermore, the compounds of Formula I in accordance with the invention, the polymeric compounds containing a repeating unit according to general Formula II or the mixtures of a plurality of the compounds according to general Formula I and/or the polymeric compounds containing a repeating unit according to general Formula II, can be produced in the form of additive compositions, for example, in the form of master batches or concentrates, which contain, for example, 10-90% of the additives in accordance with the invention, and introduced in a polymer.

In particular, the following compounds are used in accordance with the invention:

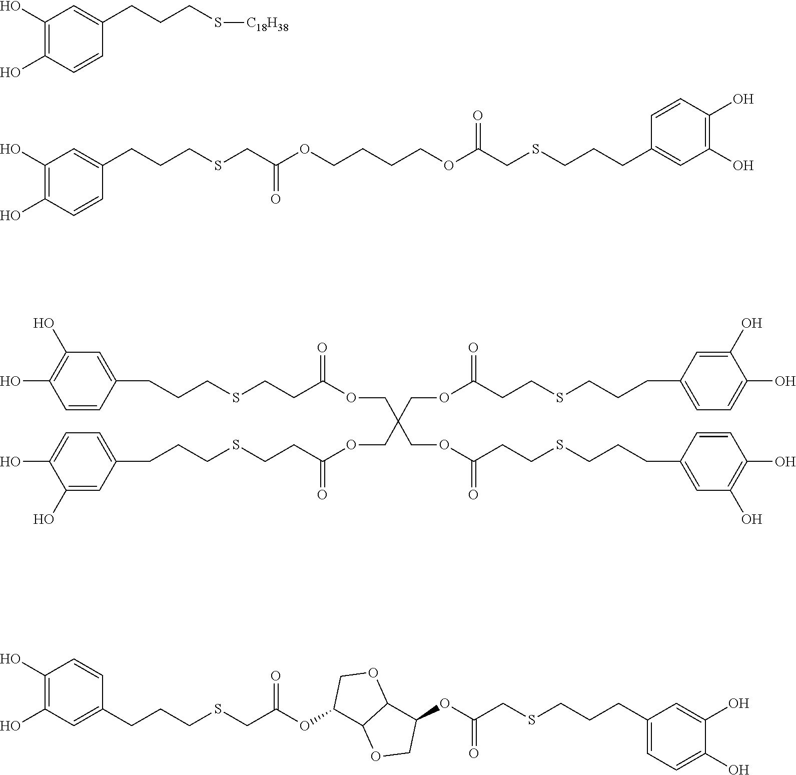

The polymeric compound containing the repeating unit according to general Formula II is preferably selected from the group consisting of homopolymers, formed from repeating units according to general Formula II, or copolymers containing the repeating unit according to general Formula II and at least one further repeating unit derived from a radical polymerizable compound, in particular repeating units derived from (meth)acrylic acid esters.

The repeating unit according to general Formula II of the polymeric compound particularly preferably has the following structure:

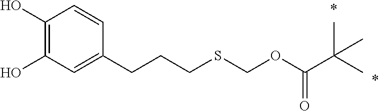

In a further preferred embodiment, all of the compounds according to general Formula I, the polymeric compounds containing a repeating unit according to general Formula II or the mixtures of a plurality of the compounds according to general Formula I and/or the polymeric compounds containing a repeating unit according to general Formula II are incorporated in the organic material at a proportion by weight of 0.01 to 10.00% by weight, preferably 0.02 to 5.00% by weight, more preferably 0.05 to 3.00% by weight, particularly preferably 0.10 to 2.00% by weight.

In the case that a plastic is stabilized, this can preferably be from the group consisting of
a) polymers from olefins or diolefins such as e.g. polyethylene (LDPE, LLDPE, VLDPE, ULDPE, MDPE, HDPE, UHMWPE), metallocene PE (m-PE), polypropylene, polyisobutylene, poly-4-methyl-pentene-1, polybutadiene, polyisoprene, such as e.g. also natural rubber (NR), polycyclooctene, polyalkylene-carbon monoxide copolymers, and copolymers in the form of random or block structures such as e.g. polypropylene-polyethylene (EP), EPM or EPDM with e.g. 5-ethylidene-2-norbornene as a comonomer, ethylene-vinyl acetate (EVA}, ethylene acrylic esters such as e.g. ethylene butyl acrylate, ethylene acrylic acid and salts thereof (ionomers), and terpolymers such as e.g. ethylene acrylic acid glycidyl(meth)acrylate, graft polymers such as e.g. polypropylene graft maleic anhydride, polypropylene graft acrylic acid, polyethylene graft-acrylic acid, polyethylene-polybutylacrylate-graft-maleic anhydride and blends such as e.g. LDPE/LLDPE or also long-chain branched polypropylene copolymers which are produced with alpha-olefins as comonomers such as e.g. with 1-butene, 1-hexene, 1-octene or 1-octadecene,
b) polystyrene, polymethylstyrene, poly-alpha-methylstyrene, polyvinylnaphthalene, polyvinylbiphenyl, polyvinyltoluene, styrene-butadiene (SB), styrene-butadiene-styrene (SBS), styrene-ethylene-butylene-styrene (SEBS), styrene-ethylene-propylene-styrene, styrene-isoprene, styrene-isoprene-styrene (SIS), styrene-butadiene-acrylonitrile (ABS), styrene-acrylonitrile (SAN), styrene-acrylonitrile-acrylate (ASA), styrene-ethylene, styrene-maleic anhydride polymers including corresponding graft copolymers such as e.g. styrene on butadiene, maleic anhydride on SBS or SEBS, and graft copolymers made of methyl methacrylate, styrene-butadiene and ABS (MABS), and hydrogenated polystyrene derivatives, such as e.g. polyvinylcyclohexane,
c) halogen-comprising polymers, such as e.g. polyvinyl chloride (PVC), polychloroprene and polyvinylidene chloride (PVDC), copolymers of vinyl chloride and vinylidene chloride or of vinyl chloride and vinyl acetate, chlorinated polyethylene, polyvinylidene fluoride, epichlorohydrin homo and copolymers in particular with ethylene oxide (ECO),
d) polymers of unsaturated esters such as polyacrylates and polymethacrylates such as polymethyl methacrylate (PMMA), polybutyl acrylate, polyauryl acrylate; poly stearyl acrylate; polyglycidyl methacrylate, polyacrylonitrile, polyacrylamides, copolymers such as polyacrylonitrile-poly alkyl acrylate,
e) polymers of unsaturated alcohols and derivatives, such as e.g. polyvinyl alcohol, polyvinyl acetate, polyvinyl butyral, polyallyl phthalate, polyallyl melamine,
f) polyacetals such as e.g. polyoxymethylene (POM) or copolymers with, for example, butanal, polyphenylene oxides and blends with polystyrene or polyamides,
g) polymers of cyclic ethers such as e.g. polyethylene glycol, polypropylene glycol, polyethylene oxide, polypropylene oxide, polytetrahydrofuran,
h) polyphenylene oxides and blends thereof with polystyrene and/or polyamides,
i) polyurethanes, made from hydroxy-terminated polyethers or polyesters and aromatic or aliphatic isocyanates such as e.g. 2,4- or 2,6-tolylene diisocyanate or methylenediphenyl diisocyanate, in particular also linear polyurethanes (TPU), polyureas,
j) polyamides such as e.g. polyamide-6, 6.6, 6.10, 4.6, 4.10, 6.12, 10.10, 10.12, 12.12, polyamide 11, polyamide 12 and (partially) aromatic polyamides such as e.g. polyphthalamides, for example, made from terephthalic acid and/or isophthalic acid and aliphatic diamines such as e.g. hexamethylenediamine or m-xylylenediamine or from aliphatic dicarboxylic acids such as e.g. adipic acid or sebacic acid and aromatic diamines such as e.g. 1,4- or 1,3-diaminobenzene, blends of different polyamides such as e.g. PA-6 and PA 6.6 or blends of polyamides and polyolefins such as e.g. PA/PP,
k) polyimides, polyamide-imides, polyetherimides, polyesterimides, poly(ether)ketones, polysulfones, polyethersulfones, polyarylsulfones, polyphenylene sulfides, polybenzimidazoles, polyhydantoins,
l) polyesters made from aliphatic or aromatic dicarboxylic acids and diols or from hydroxycarboxylic acids, such as e.g. polyethylene terephthalate (PET), polybutylene terephthalate (PBT), polypropylene terephthalate (PTT), polyethylene naphthylate (PEN), poly-1,4-dimethylolcyclohexane terephthalate, polyhydroxybenzoate, polyhydroxynaphthalate, polylactic acid (PLA), polyhydroxybutyrate (PHB), polyhydroxyvalerate (PHV), polyethylene succinate, polytetramethylene succinate, polycaprolactone,
m) polycarbonates, polyester carbonates, and blends, such as e.g. PC/ABS, PC/PBT, PC/PET/PBT, PC/PA,
n) cellulose derivatives, such as e.g. cellulose nitrate, cellulose acetate, cellulose propionate, cellulose butyrate,
o) epoxy resins, consisting of di- or polyfunctional epoxy compounds in combination with, for example, hardeners based on amines, anhydrides, dicyandiamide, mercaptans, isocyanates or catalytically active hardeners,
p) phenol resins such as e.g. phenol formaldehyde resins, urea formaldehyde resins, melamine formaldehyde resins,
q) unsaturated polyester resins from unsaturated dicarboxylic acids and diols with vinyl compounds, for example styrene, alkyd resins,
r) silicones, for example, based on dimethylsiloxanes, methyl-phenyl-siloxanes or diphenylsiloxanes, for example, terminated with vinyl groups,
s) and mixtures, combinations, or blends of two or more of the above-named polymers.

If the polymers specified under a) to r) are copolymers, these can exist in the form of statistical (“random”), block or “tapered” structures. Furthermore, the polymers mentioned can exist in the form of linear, branched, star-shaped or hyperbranched structures.

If the polymers specified under a) to r) are stereoregular polymers, they can exist in the form of isotactic, stereotactic, but also atactic forms or as stereoblock copolymers.

Furthermore, the polymers specified under a) to r) can have both amorphous and (partially) crystalline morphologies.

Optionally, the polyolefins mentioned under a) can also be crosslinked, for example crosslinked polyethylene, which is then referred to as X-PE.

Furthermore, the present compounds can be preferably used to stabilize rubbers and elastomers. This can be natural rubber (NR) or synthetic rubber materials. Suitable synthetic rubber materials consist in particular of butadiene rubber (BR), styrene-butadiene rubber (SBR), chloroprene rubber (CR), isoprene rubber (IR), isobutylene-isoprene rubber, acrylonitrile-butadiene rubber (NBR or in hydrogenated form HNBR). Further suitable rubbers and elastomers are ethylene-propylene-diene terpolymers (EPDM) and ethylene-propylene copolymers (EPM), polyester urethanes (AU), polyether urethanes (EU) and silicones (MQ).

Apart from new goods, the plastics can be recycled plastics, for example, from industrial collections such as e.g. production waste or plastics from household or recyclable collections.

Further particularly preferred are polymers from renewable raw materials, such as e.g. polylactic acid (PLA), polyhydroxybutyric acid, polyhydroxyvaleric acid, polybutylene succinate or poly(butylene succinate-co-adipate).

In addition, it is advantageous if the plastic at least one further additive, selected from the group comprising primary antioxidants, secondary antioxidants, UV absorbers, light stabilizers, metal deactivators, filler deactivators, antiozonants, nucleation agents, anti-nucleation agents, toughening agents, plasticizers, lubricants, rheological modifiers, thixotropic agents, chain extenders, optical brighteners, antimicrobial active agents (e.g. biocides), antistatic agents, slip agents, anti-blocking agents, coupling agents, crosslinking agents, branching agents, anti-cross-linking agents, hydrophilization agents, hydrophobing agents, bonding agents, dispersing agents, compatibilizers, oxygen scavengers, acid scavengers, expanding agents, degradation additives, defoaming agents, odor scavengers, marking agents, anti-fogging agents, additives to increase the electrical conductivity and/or thermal conductivity, infrared absorbers or infrared reflectors, gloss improvers, matting agents, repellents, fillers, reinforcement materials, and mixtures thereof.

Suitable primary antioxidants (A) are phenolic antioxidants, amines and lactones:

Suitable synthetic phenolic antioxidants are, for example:
alkylated monophenols, such as e.g. 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, linear or branched nonylphenols such as e.g. 2,6-dinonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof;
alkylthiomethyl phenols, such as e.g. 2,4-dioctylthiomethyl-6-tert-butylphenol, 2,4-dioctylthiomethyl-6-methylphenol, 2,4-dioctylthiomethyl-6-ethylphenol, 2,6-didodecylthiomethyl-4-nonylphenol;
hydroquinones and alkylated hydroquinones such as e.g. 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate;
tocopherols, such as e.g. α-, β-, γ-, δ-tocopherol and mixtures hereof (vitamin E);
hydroxylated thiodiphenyl ethers, such as e.g. 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide;
alkylidene bisphenols, such as e.g. 2,2′methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclhexylphenol), 2,2′-methylenebis(6-nonyl-4-methylphenol), 2,2′-methylenebis(4,6-di-tert-butylphenol), 2,2′-ethylidene bis(4,6-di-tert-butylphenol), 2,2′-ethylidene bis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol, 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethylene glycol-bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane;
O-, N- and S-benzyl compounds such as e.g. 3,5,3′,5′-tetra-tert-butyl-4,4′-dihydroxydibenzylether, cctadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate;
hydroxybenzylated malonates, such as e.g. dioctadecyl-2,2-bis(3,5-di-tert-butyl-2-hydroxybenzyl)malonate, dioctadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate;
aromatic hydroxybenzyl compounds, such as e.g. 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol;
triazine compounds, such as e.g. 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxphenylethyl)-1,3,5-triazine, 1,3,5-tris(3,5-di-tert-butyl-4-hydroxyphenylpropionyl)hexahydro-1,3,5-triazine, 1,3,5-tris(3,5-dicyclohexyl-4-hydroxybenzyl)isocyanurate;
benzyl phosphonates, such as e.g. dimethyl-2,5-di-tert-butyl-4-hydroxybenzylphosphonate, diethyl-3,5-di-tert-butyl-4-hydroxybenzylphosphonate, dioctadecyl-3,5-di-tert-butyl-4-hydroxybenzylphosphonate, dioctadecyl-5-tert-butyl-4-hydroxy-3-methylbenzylphosphonate, the calcium salt of the monoethylester of 3,5-di-tert-butyl-4-hydroxybenzylphosphonic acid;
acylaminophenols, such as e.g. 4-hydroxylauranilide, 4-hydroxystearanilide, octyl-N-(3,5-di-tert-butyl-4-hydroxyphenyl)carbamate;
esters of β-(3,5-di-tert-butyl-4-hydroxyphenyl)propionic acid with monohydric or polyhydric alcohols, for example methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
esters of β-(5-tert-butyl-4-hydroxy-3-methylphenyl)propionic acid with monohydric or polyhydric alcohols, for example methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, 3,9-bis[2-{3-(3-tert-butyl-4-hydroxy-5-methylphenyl)propionyloxy}-1,1-dimethylethyl]-2,4,8,10-tetraoxaspiro[5.5]undecane; esters of β-(3,5-dicyclohexyl-4-hydroxyphenyl)propionic acid with monohydric or polyhydric alcohols, for example methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
esters of (3,5-di-tert-butyl-4-hydroxyphenyl)acetic acid with monohydric or polyhydric alcohols, for example methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
amides of β-(3,5-di-tert-butyl-4-hydroxyphenyl)propionic acid, such as e.g. N,N′-bis(3,5-di-tert-butyl-4-hydroxyphenylpropionyl)hexamethylene diamide, N,N′-bis(3,5-di-tert-butyl-4-hydroxyphenylpropionyl)hexamethylene diamide, N,N′-bis(3,5-di-tert-butyl-4-hydroxyphenylpropionyl)hexamethylene diamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1, marketed by Uniroyal);
ascorbic acid (vitamin C).

Particularly preferred phenolic antioxidants are the following structures:

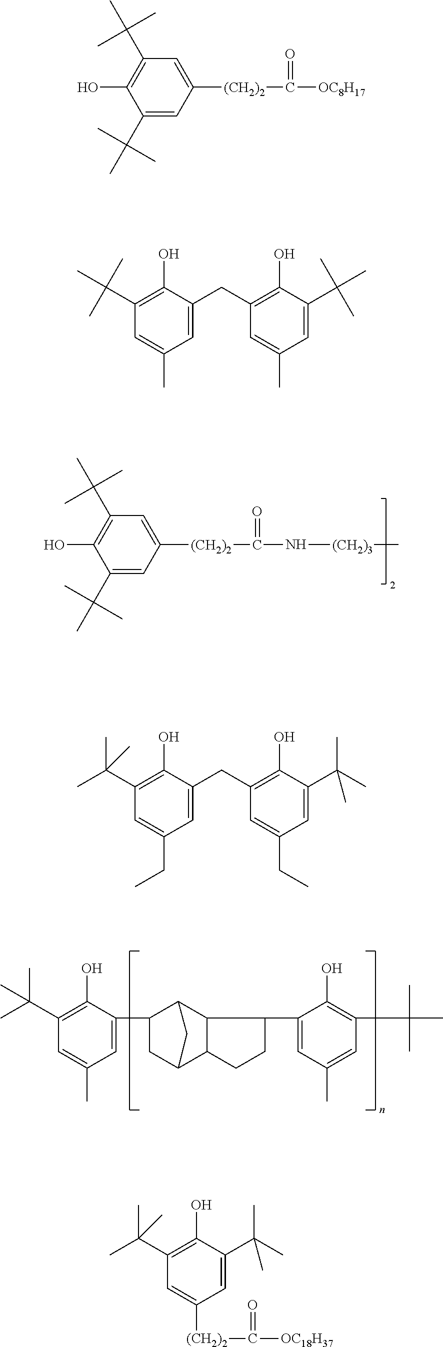

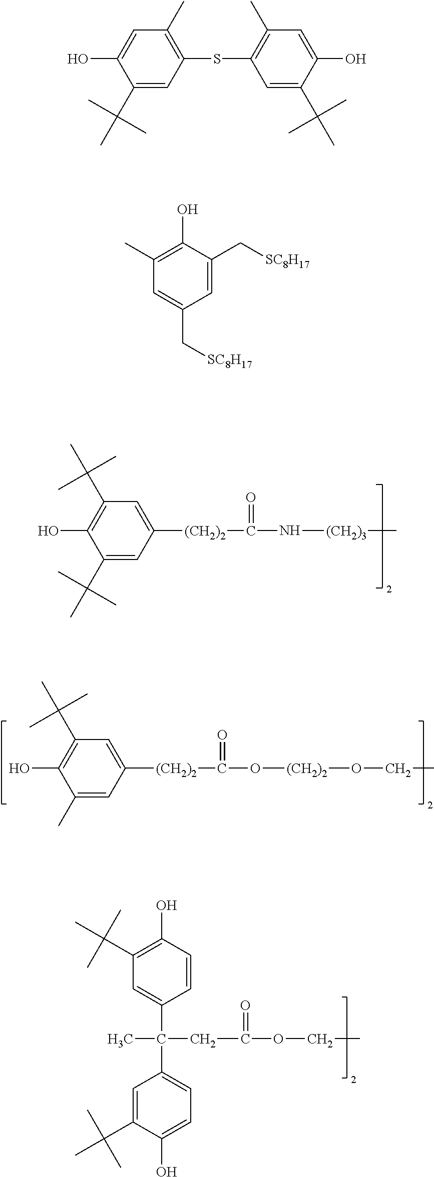

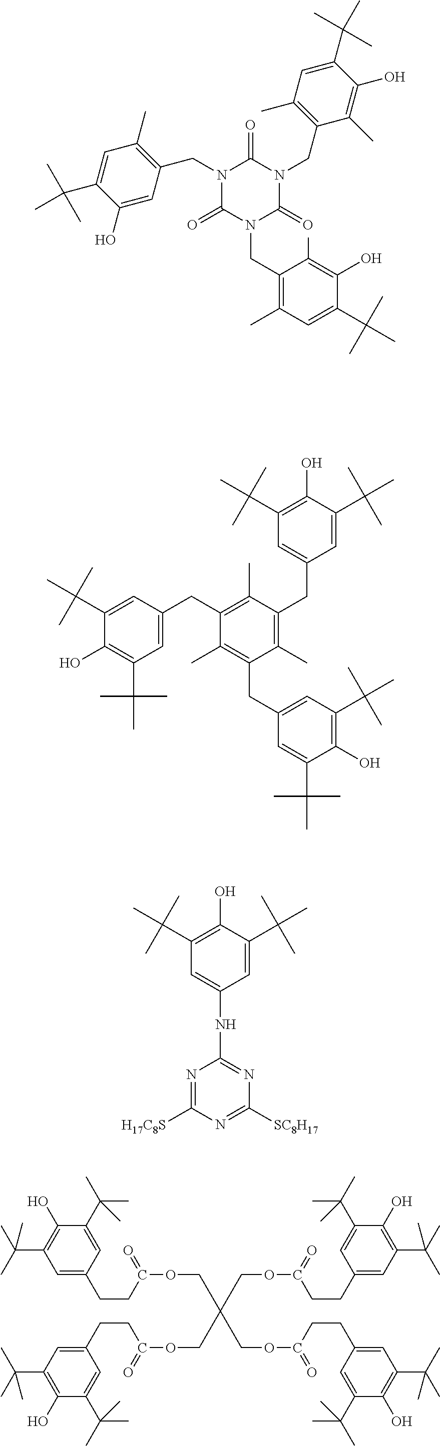

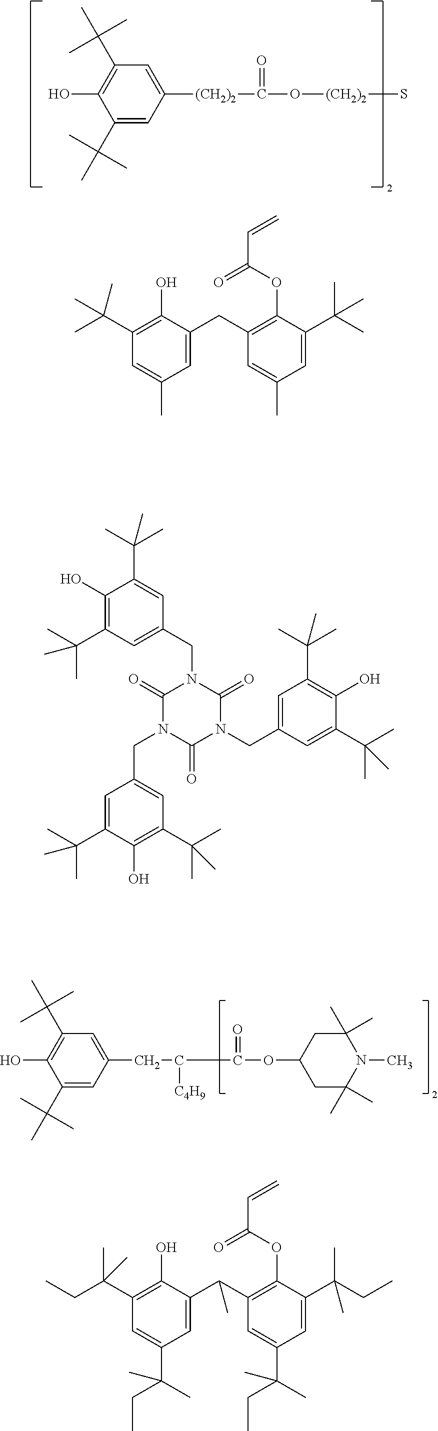

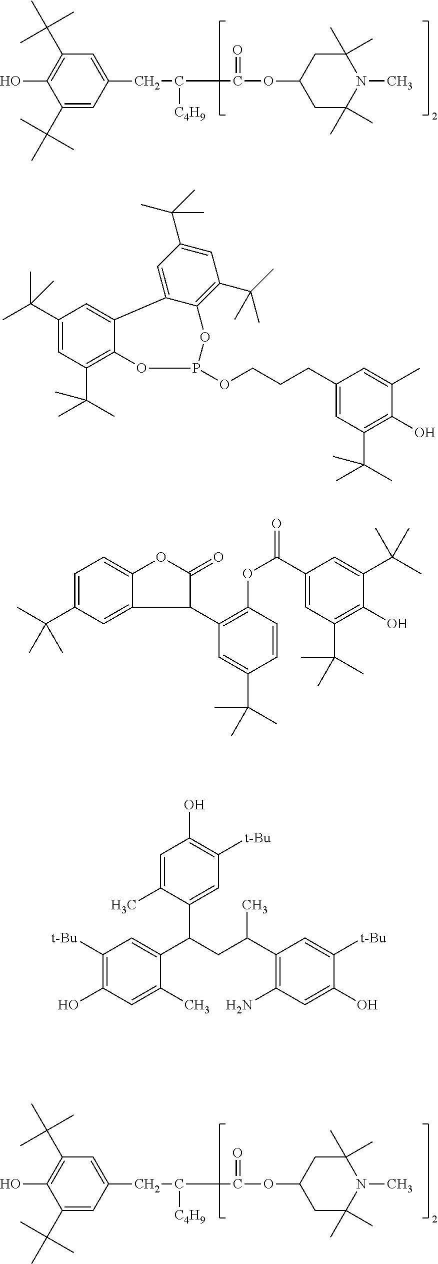

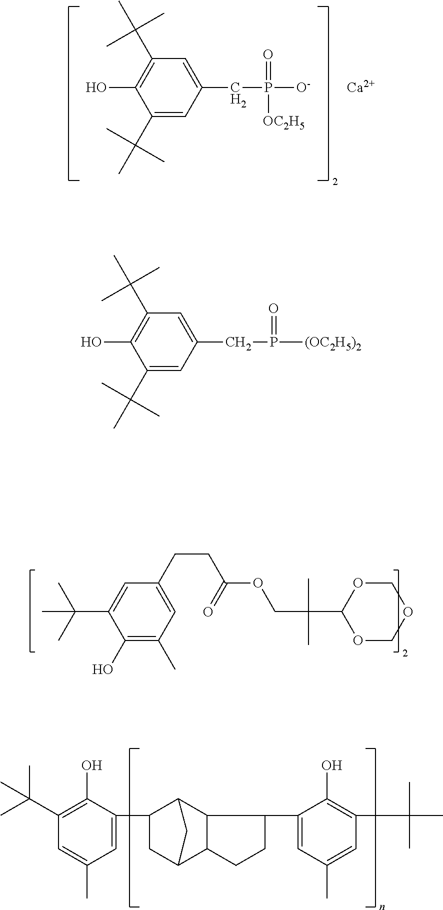

Very particularly preferred phenolic antioxidants are octadecyl-3-(3,5-di-tert-butyl-4-hydroxyphenyl)propionate and pentaerythritol tetrakis(3-(3,5-di-tert-butyl-4-hydroxyphenyl)propionate).

Further particularly preferred phenolic antioxidants are based on renewable raw materials such as e.g. tocopherols (vitamin E), tocotrienols, tocomonoenols, carotenoids, hydroxytyrosol, flavonols such as e.g. chrysin, quercetin, hesperidin, neohesperidin, naringin, morin, camphor oil, fisetin, anthocyanins such as e.g. delphinidin and malvidin, curcumin, carnosic acid, carnosol, rosmarinic acid, and resveratrol.

Suitable aminic antioxidants are, for example:
N,N′-di-isopropyl-p-phenylene diamine, N,N′-di-sec-butyl-p-phenylene diamine, N,N′-bis(1,4-dimethylpentyl)-p-phenylene diamine, N,N′-bis(1-ethyl-3-methylpentyl)-p-phenylene diamine, N,N′-bis(1-methylheptyl)-p-phenylene diamine, N,N′-dicyclohexyl-p-phenylene diamine, N,N′-diphenyl-p-phenylene diamine, N,N′-bis(2-naphthyl)-p-phenylene diamine, N-isopropyl-N′-phenyl-p-phenylene diamine, N-(1,3-dimethylbutyl)-N′-phenyl-p-phenylene diamine, N-(1-methylheptyl)-N′-phenyl-p-phenylene diamine, N-cyclohexyl-N′-phenyl-p-phenylene diamine, 4-(p-toluene sulfamoyl)diphenylamine, N,N′-dimethyl-N,N′-di-sec-butyl-p-phenylene diamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylamino-phenol, bis(4-methoxyphenyl)amine, 2,6-di-tert-butyl-4-dimethylaminomethyl-phenol, 2,4′-diaminodiphenylmethane, 4,4′-diaminodiphenylmethane, N,N,N′,N′-tetra-methyl-4,4′-diaminodiphenylmethane, 1,2-bis[(2-methyl-phenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyl-diphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazinene, N-allylphenothiazine, N,N,N′,N′-tetraphenyl-1,4-diaminobut-2-ene and mixtures or combinations hereof.

Preferred aminic antioxidants are: N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine.

Particularly preferred aminic antioxidants are the structures:

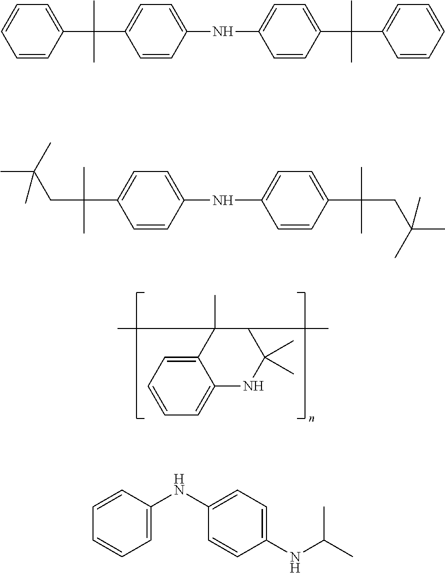

Further preferred aminic antioxidants are hydroxylamines or N-oxides (nitrones), such as e.g. N,N-dialkyl hydroxylamines, N,N-dibenzyl hydroxylamine, N,N-dilauryl hydroxylamine, N,N-distearyl hydroxylamine, N-benzyl-α-phenylnitrone, N-octadecyl-α-hexadecylnitrone, and Genox EP (SI Group) according to the formula:

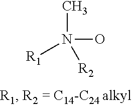

Suitable lactones are benzofuranones and indolinones such as e.g. 3-(4-(2-acetoxyethoxy)phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-(2-hydroxyethoxy]phenyl)benzofuran-2-one), 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, 3-(2,3-dimethylphenyl)-5,7-di-tert-butyl-benzofuran-2-one as well as lactones that additionally contain a phosphite group such as e.g.

A further suitable group of antioxidants are isoindolo[2,1-A]quinazolines such as e.g.

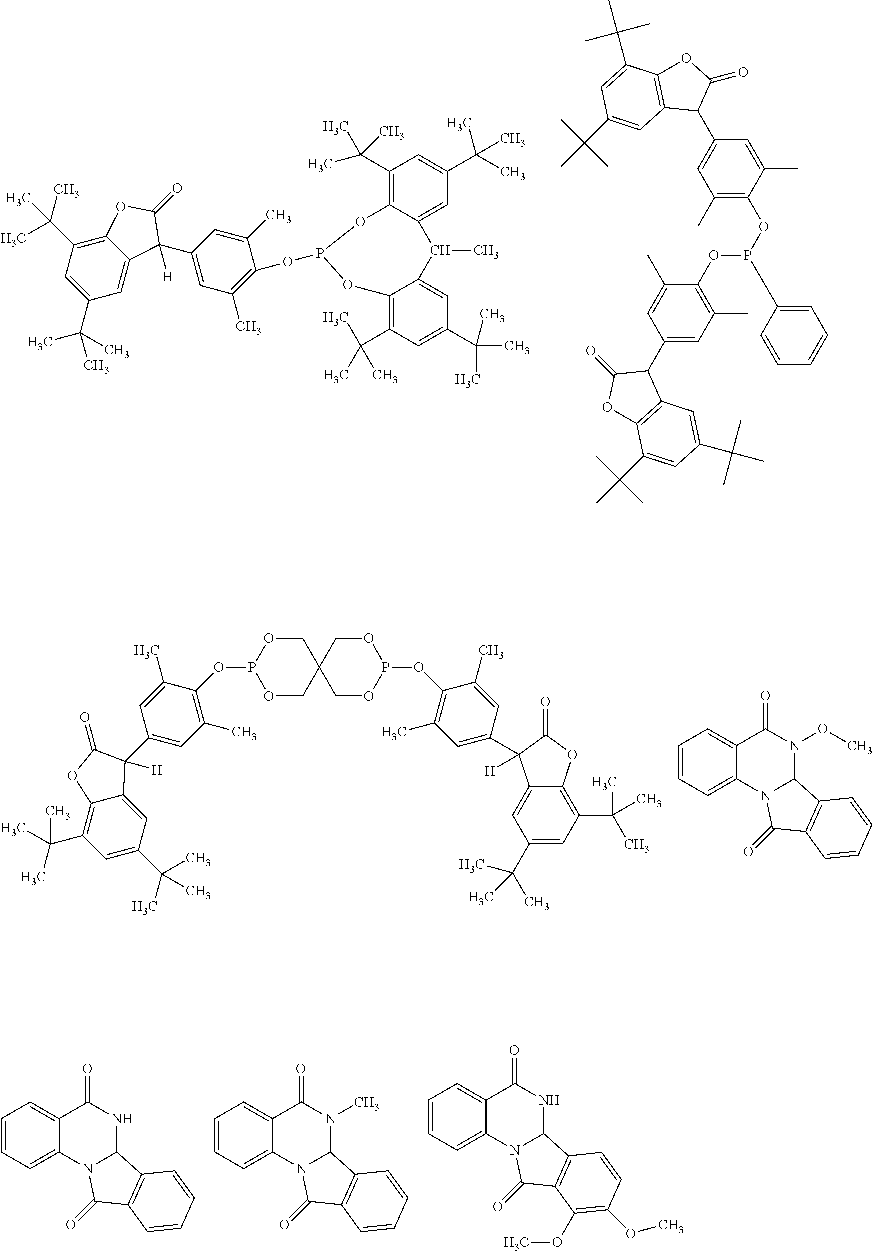

Suitable secondary antioxidants are in particular phosphites or phosphonites such as
triphenylphosphite, diphenylalkylphosphite, phenyldialkylphosphite, tri(nonylphenyl)phosphite, trilaurylphosphite, trioctadecylphosphite, distearylpentaerythritoldiphosphite, tris-(2,4-di-tert-butylphenyl)phosphite, diisodecylpentaerythritoldiphosphite, bis(2,4-di-tert-butylphenyl)pentaerythritoldiphosphite, bis(2,4-di-cumylphenyl)pentaerythritoldiphosphite, bis(2,6-di-tert-butyl-4-methylphenyl)pentaerythritoldiphosphite, diisodecyloxypentaerythritoldiphosphite, bis(2,4-di-tert-butyl-6-methylphenyl)pentaerythritoldiphosphite, bis(2,4,6-tris(tert-butylphenyl)pentaerythritoldiphosphite, tristearylsorbitoltriphosphite, tetrakis(2,4-di-tert-butylphenyl)-4,4′-biphenylene diphosphonite, 6-isooctyloxy-2,4,8,10-tetra-tert-butyl-12H-dibenz[d,g]-1,3,2-dioxaphosphocine, bis(2,4-di-tert-butyl-6-methylphenyl)methylphosphite, bis(2,4-di-tert-butyl-6-methylphenyl)ethylphosphite, 6-fluoro-2,4,8,10-tetra-tert-butyl-12-methyl-dibenz[d,g]-1,3,2-dioxaphosphocine, 2,2′2″-nitrilo[triethyltris(3,3″,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl))phosphite, 5-butyl-5-ethyl-2-(2,4,6-tri-tert-butylphenoxy)-1,3,2-dioxaphosphirane.

Particularly preferred phosphites are:

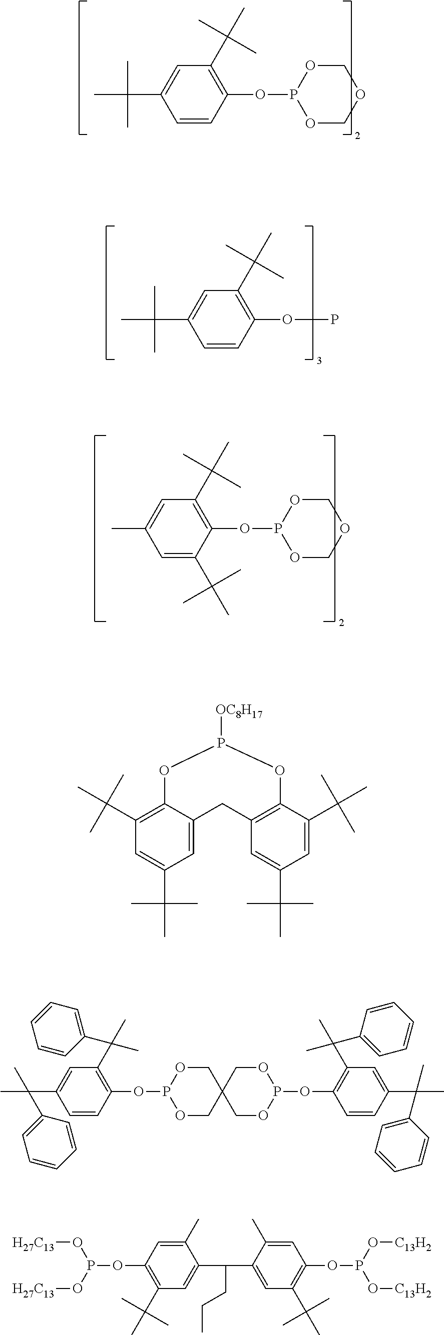

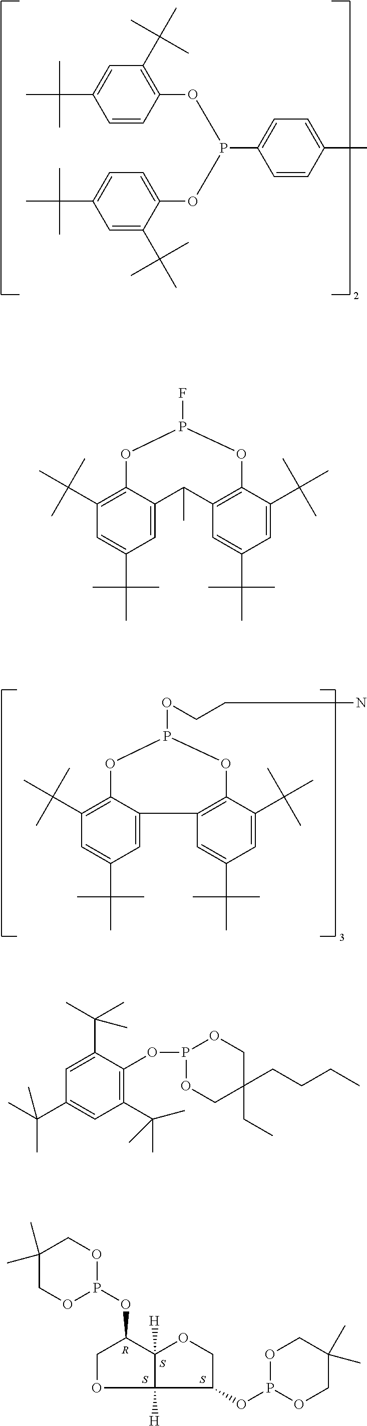

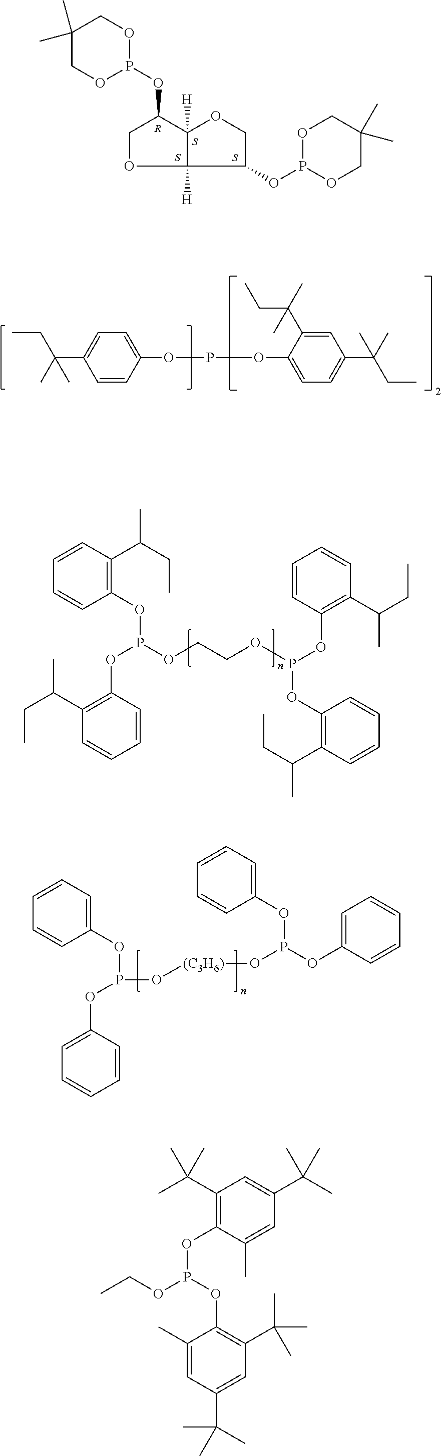

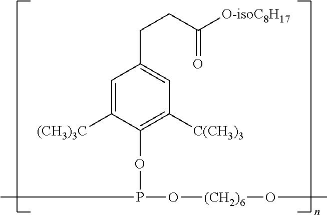

with n=3-100

A preferred phosphonite is:

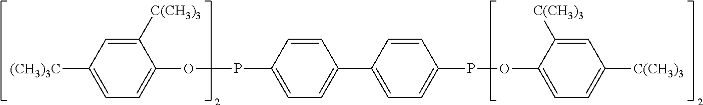

Very Particularly preferably, the phosphite tris-(2,4-di-tert-butylphenyl)phosphite is used as the secondary antioxidant.

Suitable secondary antioxidants are furthermore organosulfur compounds such as e.g. sulfides and disulfides, e.g. distearyl thiodipropionate, dilauryl thiodipropionate; ditridecyldithiopropionate, ditetradecyl thiodipropionate, 3-(dodecylthio)-1,1′-[2,2-bis[[3-(dodecylthio)-1-oxopropoxy]methyl]-1,3-propanediyl] propanoic acid ester. The following structures are preferred:

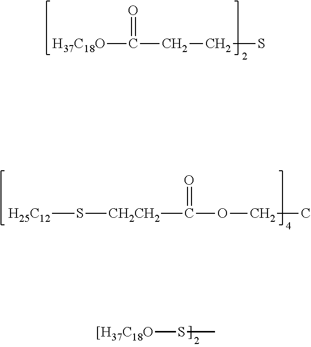

Suitable acid scavengers (“antacids”) are salts of monovalent, bivalent, trivalent, or quadrivalent metals, preferably alkali metals, alkaline earth metals, aluminum or zinc, in particular formed with fatty acids such as e.g. calcium stearate, magnesium stearate, zinc stearate, aluminum stearate, calcium laurate, calcium behenate, calcium lactate, calcium stearolyl-2-lactate. Further classes of suitable acid scavengers are hydrolactites, in particular synthetic hydrolactites on the basis of aluminum, magnesium and zinc, hydrocalumites, zeolites, alkaline earth oxides, in particular calcium oxide and magnesium oxide and zinc oxide, alkaline earth carbonates, in particular calcium carbonate, magnesium carbonate, dolomite, and hydroxides, in particular brucite (magnesium hydroxide),

Suitable costabilizers are furthermore polyols, in particular alditols or cyclitols. Polyols are, for example, pentaerythritol, dipentaerythritol, tripentaerythritol, short-chain polyether polyols or polyester polyols, and hyperbranched polymers/oligomers or dendrimers having alcohol groups, for example:

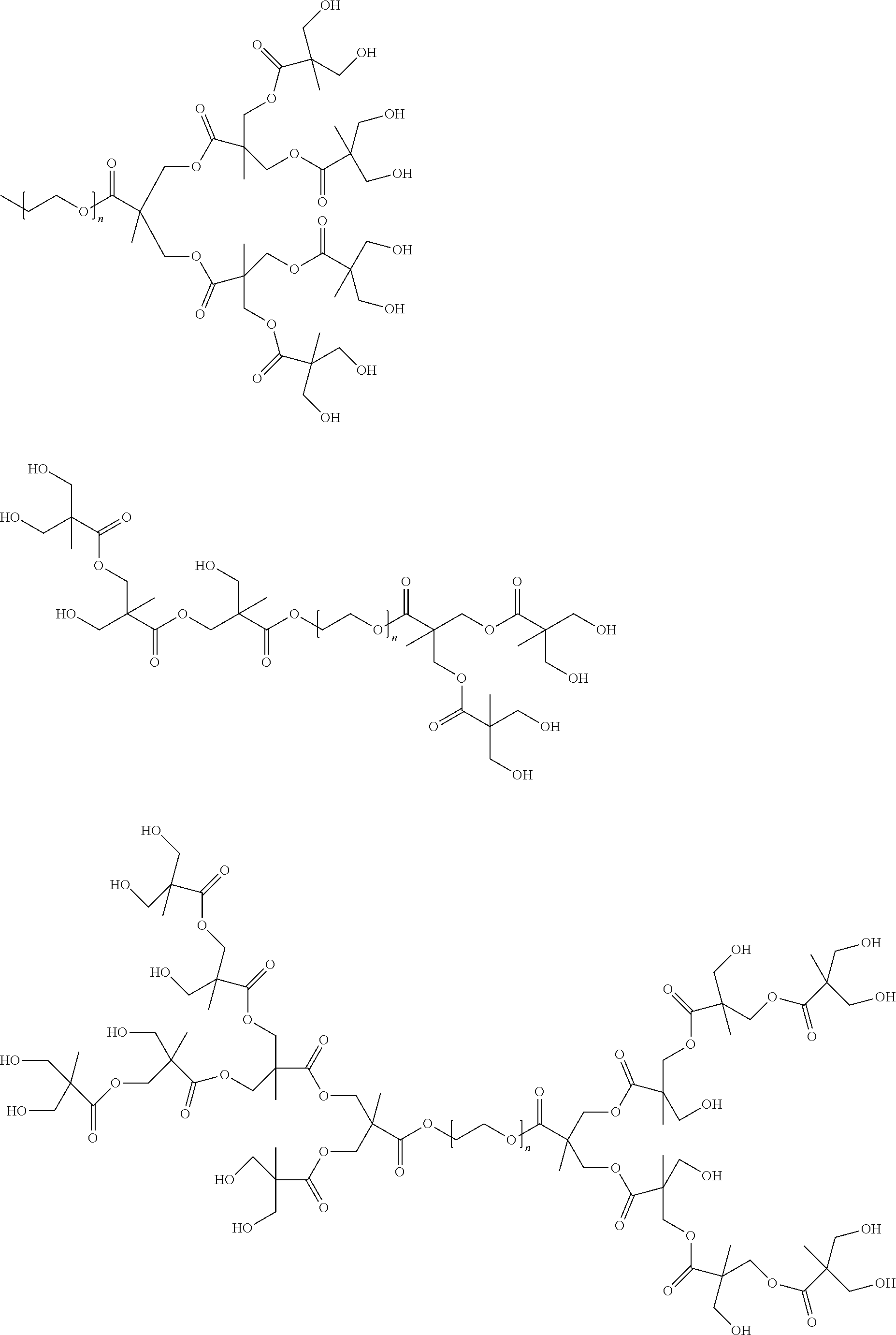

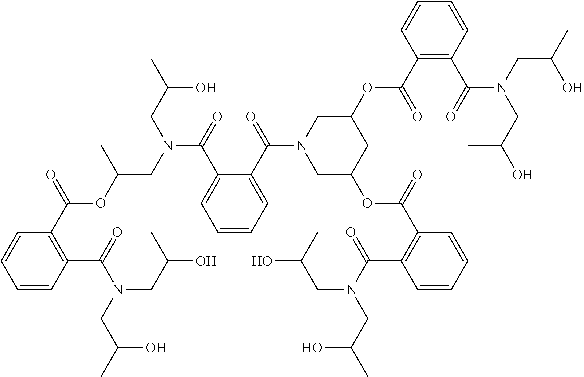 [SMILES: CC(O)CN(CC(C)O)C(=O)c1ccccc1C(=O)OC(C)CN(CC(C)O)C(=O)c1ccccc1C(=O)N1CC(OC(=O)c2ccccc2C(=O)N(CC(C)O)CC(C)O)CC(OC(=O)c2ccccc2C(=O)N(CC(C)O)CC(C)O)C1]

The at least one alditol is preferably selected from the group consisting of threitol, erythritol, galactitol, mannitol, ribitol, sorbitol, xylitol, arabitol, isomalt, lactitol, maltitol, altritol, iditol, maltotritol and hydrogenated oligo- and polysaccharides having polyol end groups and mixtures thereof. The at least one preferred alditol is particularly preferably selected from the group comprising erythritol, mannitol, isomaltol, maltitol, and mixtures thereof.

Examples of further suitable sugar alcohols are heptitols and octitols: meso-glycero-allo-heptitol, D-glycero-D-altro-heptitol, D-glycero-D-manno-heptitol, meso-glycero-gulo-heptitol, D-glycero-D-galacto-heptitol (perseitol), D-glycero-D-gluco-heptitol, L-glycero-D-gluco heptitol, D-erythro-L-galacto-octitol, D-threo-L-galacto-octitol.

The at least one cyclitol can in particular be selected from the group consisting of inositol (myo, scyllo-, D-chiro-, L-chiro-, muco-, neo-, allo-, epi- and cis-inositol), 1,2,3,4-tetra hydroxycyclohexane, 1,2,3,4,5-pentahydroxy cyclohexane, quercitol, viscumitol, bornesitol, conduritol, ononitol, pinitol, pinpollitol, quebrachitol, ciceritol, quinic acid, shikimic acid, and valienol, with myo-inositol (myo-inositol) being preferred here.

Suitable light stabilizers are, for example, compounds based on 2-(2′-hydroxyphenyl)benzotriazoles, 2-hydroxybenzophenones, esters of benzoic acids, acrylates, oxamides and 2-(2-hydroxyphenyl)-1,3,5-triazines.

Suitable 2-(2′-hydroxyphenyl)benzotriazoles are, for example, 2-(2′-hydroxy-5′methylphenyl)benzotriazole, 2-(3′,5′-di-tert-butyl-2′-hydroxyphenyl)benzotriazole, 2-(5′-tert-butyl-2′-hydroxy-phenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl-5-chlorobenzotriazole, 2-(3′-sec-butyl-5′-tert-butyl-2′-hydroxy-phenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis(α,α-dimethylbenzyl)-2′-hydroxyphenyl)benzotriazole, 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxyphenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[4-(1,1,3,3-tetramethylbutyl)-6-benzotriazole-2-ylphenol]; the product of the transesterification of 2-[3′-tert-butyl-5′-(2-methoxycarbonylethyl)-2′-hydroxyphenyl]-2H-benzotriazole with polyethylene glycol 300; [R—CH2CH2-COO—CH2CH2-]-2, where R=3′-tert-butyl-4′-hydroxy-5′-2H-benzotriazole-2-ylphenyl, 2-[2′-hydroxy-3′-(α,α-dimethylbenzyl)-5′-(1,1,3,3-tetramethylbutyl)phenyl]benzotriazole, 2-[2′-hydroxy-3′-(1,1,3,3-tetramethylbutyl)-5′-(α,α-dimethylbenzyl)phenyl]benzotriazole.

Suitable 2-hydroxybenzophenones are, for example, 4-hydroxy-, 4-methoxy-, 4-octyloxy-, 4-decyloxy-4-dodecyloxy, 4-benzyloxy, 4,2′,4′-trihydroxy- and 2′-hydroxy-4,4′-dimethyoxy derivatives of the 2-hydroxy benzophenones.

Suitable acrylates are, for example, ethyl-α-cyano-β,β-diphenylacrylate, isooctyl-α-cyano-β,β-diphenylacrylate, methyl-α-carbomethoxycinnamate, methyl-α-cyano-β-methyl-p-methoxycinnamate, butyl-α-cyano-β-methyl-p-methoxycinnamate, methyl-α-carbomethoxy-p-methoxycinnamate and N-(β-carbomethoxy-β-cyanovinyl)-2-methylindoline.

Suitable esters of benzoic acids are, for example, 4-tert-butylphenylsalicylate, phenylsalicylate, octylphenylsalicylate, dibenzoylresorcinol, bis(4-tert-butylbenzoyl)resorcinol, benzoylresorcinol, 2,4-di-tert-butylphenyl-3,5-di-tert-butyl-4-hydroxybenzoate, hexadecyl-3,5-di-tert-butyl-4-hydroxybenzoate, octadecyl-3,5-di-tert-butyl-4-hydroxybenzoate, 2-methyl-4,6-di-tert-butylphenyl-3,5-di-tert-butyl-4-hydroxybenzoate.

Suitable oxamides are, for example, 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixtures with 2-ethoxy-2′-ethyl-5,4′-di-tert-butoxanilide, mixtures of o- and p-methoxy-disubstituted oxanilides and mixtures of o- and p-ethoxy-disubstituted oxanilides.

Suitable 2-(2-hydroxyphenyl)-1,3,5-triazines are, for example, 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)-phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl-1,3,5-triazine.

Suitable metal deactivators are, for example, N,N′-diphenyloxamide, N-salicylal-N′-salicyloylhydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyldihydrazide, oxanilide, isophthaloyldihydrazide, sebacoyl-bis-phenylhydrazide, N,N′-diacetyladipoyldihydrazide, N,N′-bis(salicyloyl)oxylyldihydrazide, N,N′-bis(salicyloyl)thiopropionyldihydrazide.

Particularly preferred as metal deactivators are:

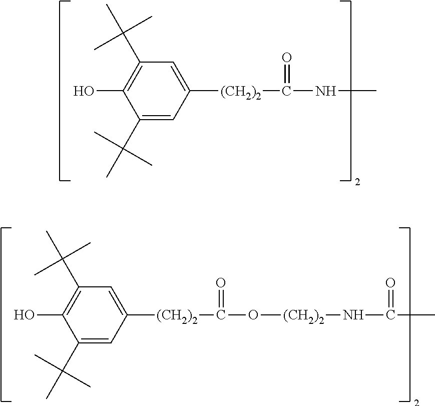

Suitable hindered amines are, for example, 1,1-bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl)-n-butyl-3,5-di-tert-butyl-4-hydroxybenzylmalonate, the condensation product of 1-(2-hydroxyethyl)-2,2,6,6-tetramethyl-4-hydroxypiperidine and succinic acid, linear or cyclic condensation products of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butanetetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, linear or cyclic condensation products from N,N′-bis(2,2,6,6-tetramethyl-4-piperidyl)hexamethylenediamine and 4-morpholino-2,6-dichloro-1,3,5-triazine the reaction product of 7,7,9,9-tetramethyl-2-cycloundecyl-1-oxa-3,8-diaza-4-oxospiro-[4,5]decane and epichlorohydrin.

The sterically hindered N—H, N-alkyl such as N-methyl or N-octyl, the N-alkoxy derivatives such as N-methoxy or N-octyloxy, the cycloalkyl derivatives such as N-cyclohexyloxy and the N-(2-hydroxy-2-methylpropoxy) analogs are also each included in the above-given structures here.

Preferred hindered amines also have the following structures:

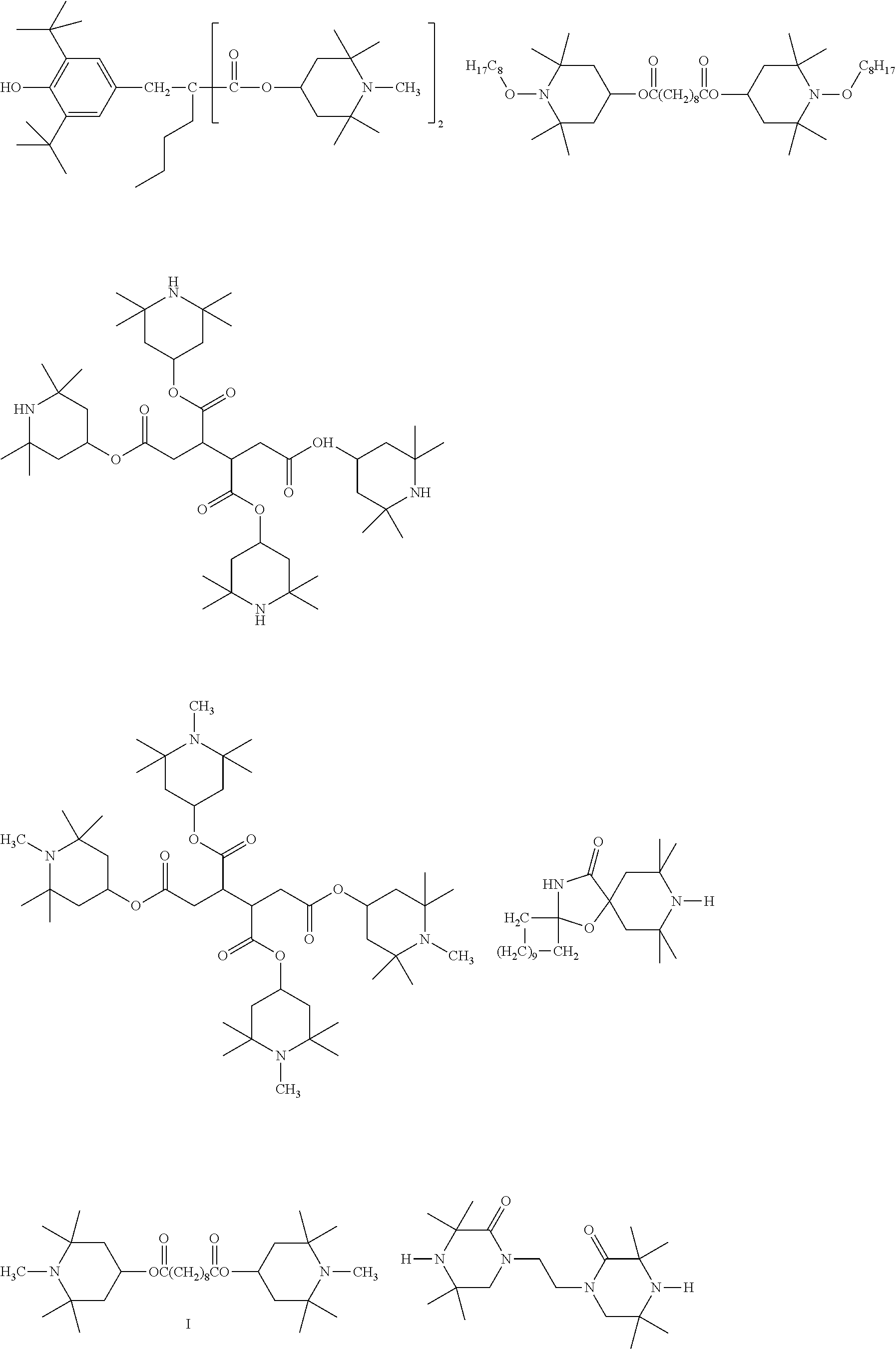

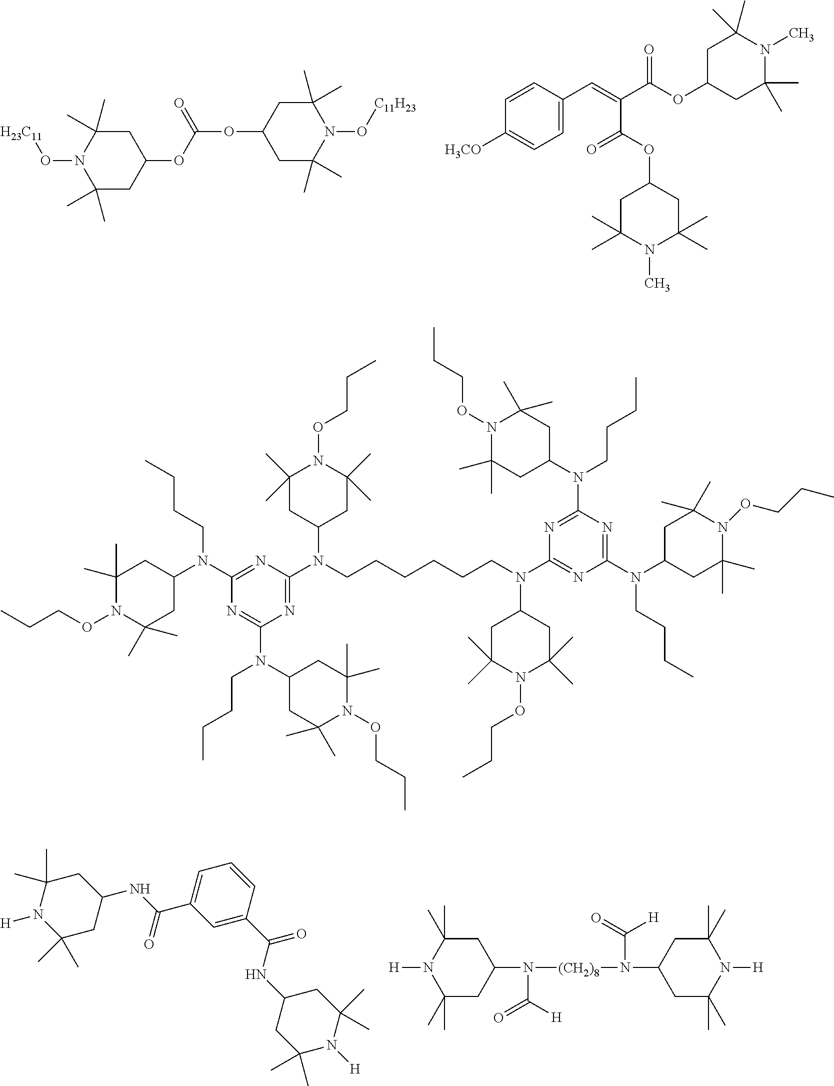

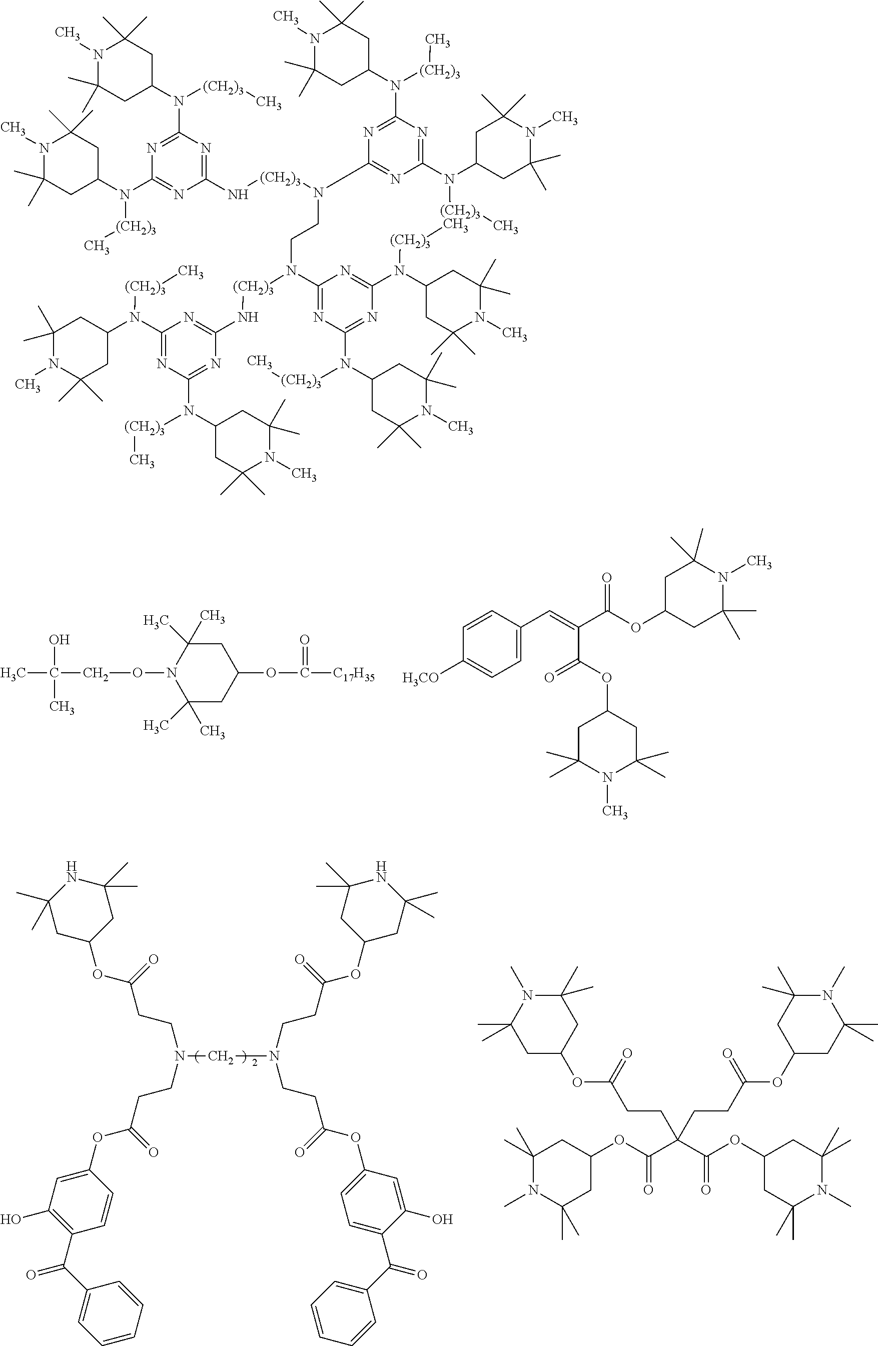

Preferred oligomeric and polymeric hindered amines have the following structures:

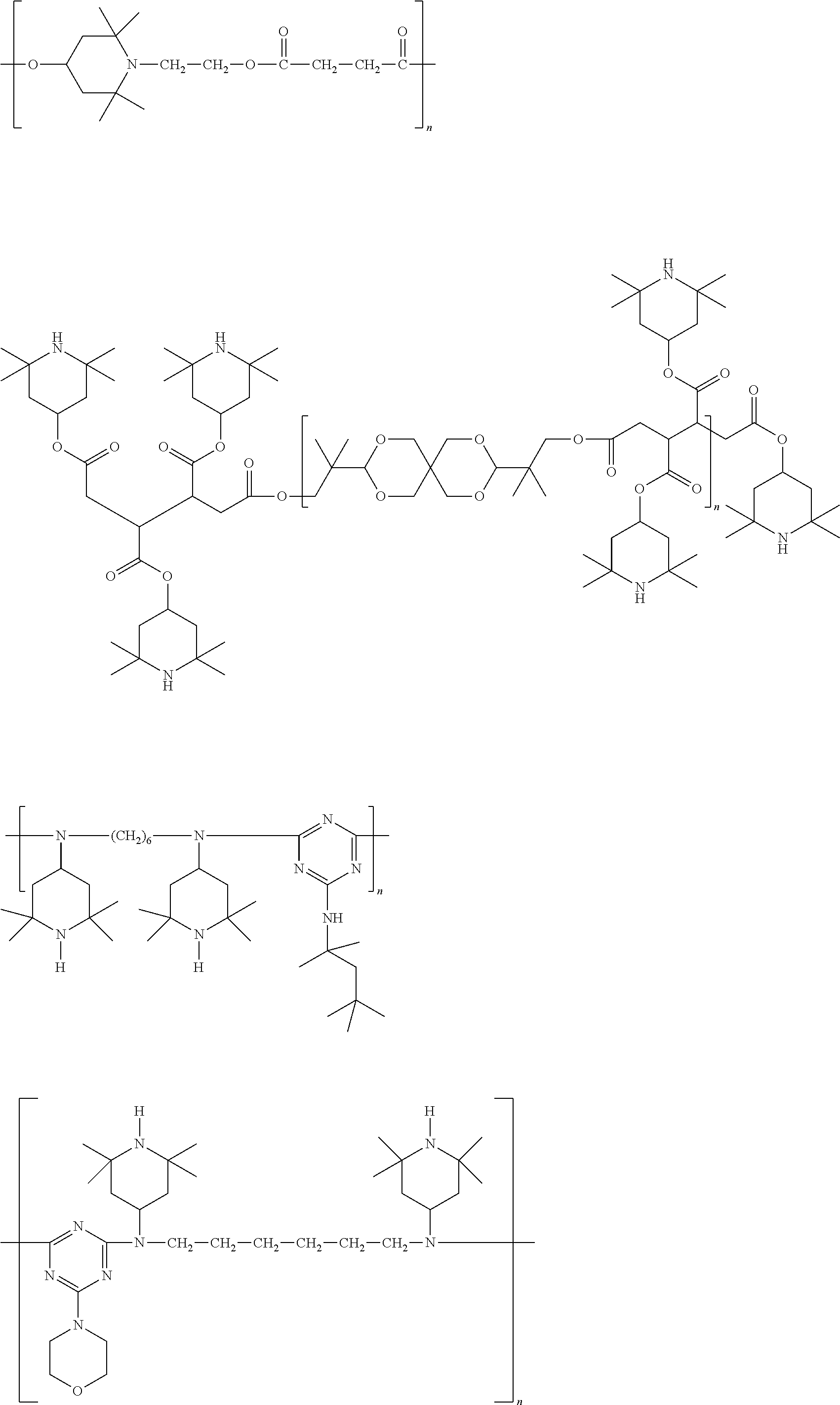

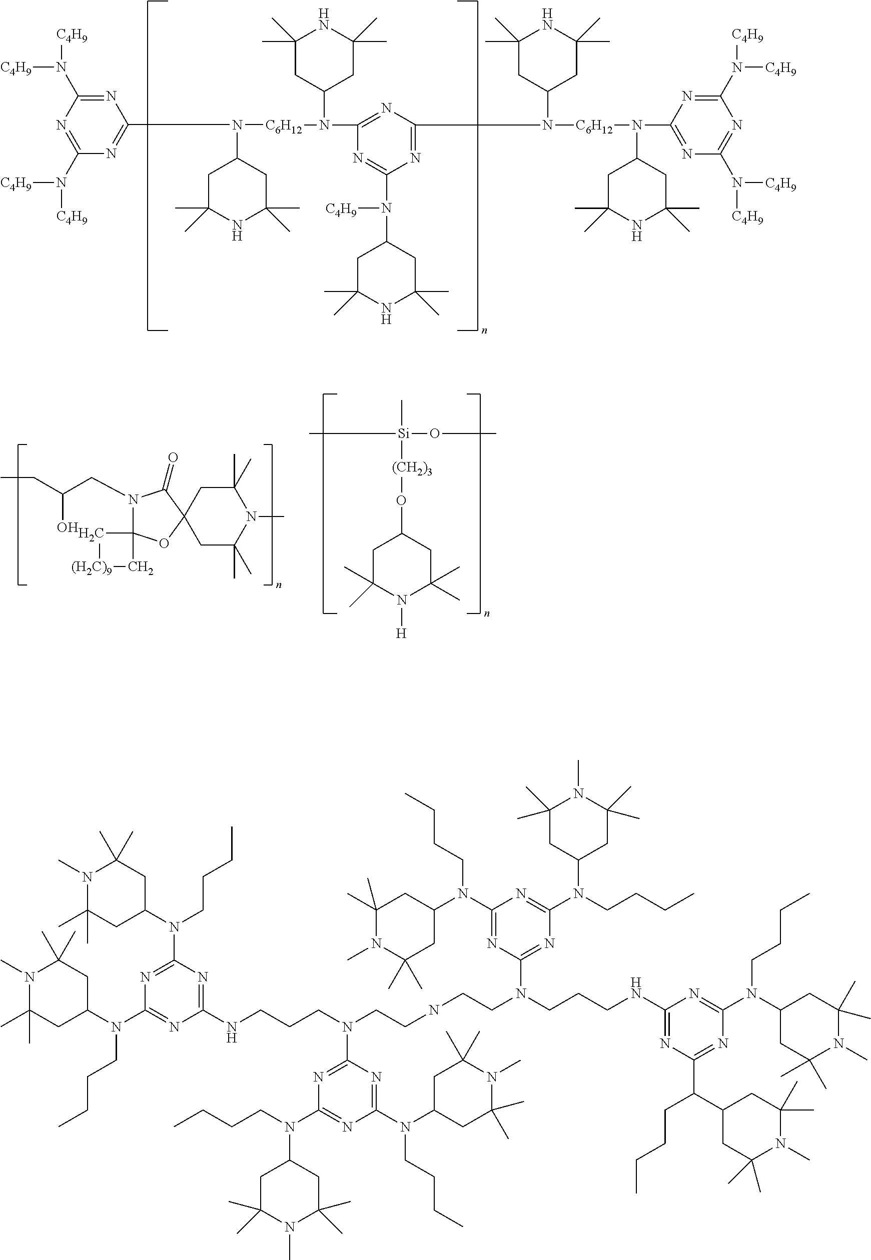

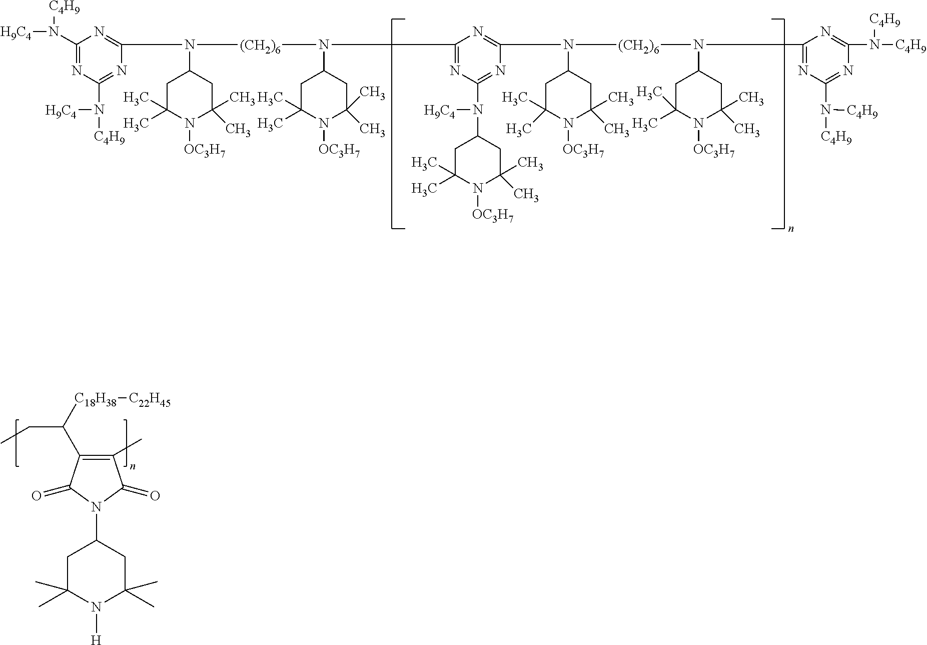

In the above-named compounds, n respectively means 3 to 100.

A further suitable light stabilizer is Hostanox NOW (manufacturer: Clariant SE) with the following general structure:

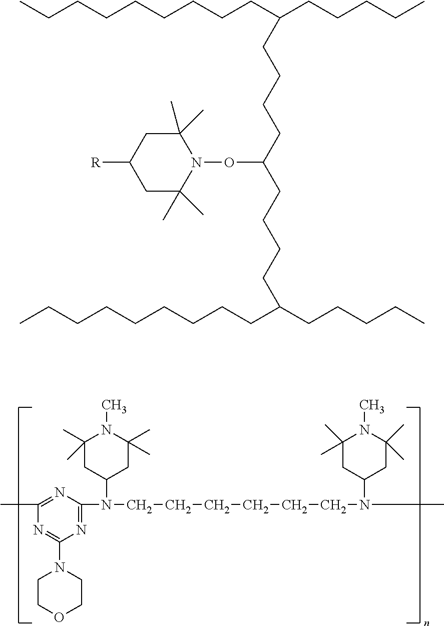

where R means —O—C(O)—C₁₅H₃₁ or —O—C(O)—C₁₇H₃₅.

Suitable dispersants are, for example:
polyacrylates, for example, copolymers having long chain side groups, polyacrylate block copolymers, alkylamides: for example, N,N′-1,2-ethanediylbis octadecanamide sorbitan esters, for example, monostearyl sorbitan esters, titanates and zirconates, reactive copolymers having functional groups, for example, polypropylene-co-acrylic acid, polypropylene-co-maleic anhydride, polyethylene-co-glycidyl methacrylate, polystyrene-alt-maleic anhydride-polysiloxanes: for example, dimethylsilanediol-ethylene oxide copolymer, polyphenylsiloxane copolymer, amphiphilic copolymers: for example, polyethylene block polyethylene oxide, dendrimers, for example, dendrimers comprising hydroxyl groups.

Suitable antinucleation agents are azine dyes such as e.g. nigrosin.

Suitable flame retardant agents are, in particular:
inorganic flame retardant agents such as Al(OH)₃, Mg(OH)₂, AlO(OH), MgCO₃, sheet silicates such as montmorillonite or sepiolite, non-modified or organically modified double salts such as Mg—Al silicates, POSS (polyhedral oligomeric silsesquioxane) compounds, huntite, hydro magnesite or halloysite and Sb₂O₃, Sb₂O₅, MoO₃, zinc stannate, zinc hydroxystannate,
flameproofing agents comprising nitrogen such as e.g. melamine, melem, melam, melon, melamine derivatives, melamine condensation products or melamine salts, benzoguanamine, polyisocyanurates, allantoin, phosphacenes, in particular melamine cyanurate, melamine phosphate, dimelamine phosphate, melamine pyrophosphate, melamine polyphosphate, melamine metal phosphates such as e.g. melamine aluminum phosphate, melamine zinc phosphate, melamine magnesium phosphate, and the corresponding pyrophosphates and polyphosphates, poly-[2,4-(piperazine-1,4-yl)-6-(morpholine-4-yl)-1,3,5-triazine], ammonium polyphosphate, melamine borate, melamine hydrobromide,
radical formers, such as alkoxyamines, hydroxylamine esters, azo compounds, sulfene amides, sulfene imides, dicumyl or polycumyl, hydroxyimide and derivatives thereof such as e.g. hydroxyimide esters or hydroxyimide ethers,
flame retardant agents containing phosphorus such as e.g. red phosphorus, phosphates such as e.g. resorcin diphosphate, bisphenol-A-diphosphate, and their oligomers, triphenylphosphate, ethylene diamine diphosphate, phosphinates such as e.g. hypophosphorous acid and their derivatives such as alkylphosphinate salts, e.g. diethylphosphinate aluminum or diethylphosphinate zinc or aluminum phosphinate, aluminum phosphite, aluminum phosphonate, phosphonate esters, oligomer and polymer derivatives of the methane phosphonic acid, 9,10-dihydro-9-oxa-10-phosphorylphenanthrene-10-oxide (DOPO) and their substituted compounds,
halogenated flameproofing agents based on chlorine and bromine, such as e.g. polybrominated diphenyl oxides, such as e.g. decabromodiphenyloxide, tris(3-bromo-2,2-bis (bromomethyl)propyl-phosphate, tris(tribromoneopentyl)phosphate, tetrabromophthalic acid, 1,2-bis(tribromophenoxy)ethane, hexabromocyclododecane, brominated diphenylethane, tris-(2,3-dibromopropyl)isocyanurate, ethylene-bis-(tetrabromophthalimide), tetrabromo-bisphenol A, brominated polystyrene, brominated polybutadiene or polystyrene brominated polybutadiene copolymers, brominated polyphenylene ether, brominated epoxy resin, polypentabromobenzylacrylate, optionally in combination with Sb2O3 and/or Sb2O5,
borates, such as e.g. zinc borate or calcium borate, optionally on a carrier material, such as e.g. silica,
sulphur-comprising compounds, such as e.g. elemental sulphur, disulfides and polysulfides,
thiuram sulfide, dithiocarbamates, mercaptobenzothiazole and sulfenamides,
anti-drip agents, such as e.g. polytetrafluoroethylene,
silicon-comprising compounds, such as e.g. polyphenyl siloxanes,
carbon modifications, such as e.g. carbon nanotubes (CNTs), expandable graphite or graphene,
and combinations or mixtures thereof.

Suitable plasticizers are, for example, phthalic acid esters, adipic acid esters, esters of citric acid, esters of 1,2-cyclohexane dicarboxylic acid, trimellitic acid esters, isorobide esters, phosphate esters, epoxides such as e.g. epoxidized soy bean oil, or aliphatic polyesters.

Suitable mold lubricants and processing aids are, for example, polyethylene waxes, polypropylene waxes, salts of fatty acids such as e.g. calcium stearate, zinc stearate, or salts of montane waxes, amide waxes such as e.g. erucic acid amide or oleic acid amides, fluoropolymers, silicones, or neoalkoxytitanates and zirconates.

Suitable pigments may be inorganic or organic. Inorganic pigments are, for example, titanium dioxide, zinc oxide, zinc sulfide, iron oxide, ultramarine, carbon black, organic pigments are, for example, anthraquinones, anthanthrones, benzimidazolones, quinacridones, diketopyrrolopyrroles, dioxazines, indanthrones, isoindolinones, azo compounds, perylenes, phthalocyanines or pyranthrones. Further suitable pigments include effect pigments on a metal basis or pearl gloss pigments on a metal oxide basis.

Suitable optical brighteners are, for example, bis-benzoxazoles, phenylcumarines, or bis(styryl)biphenyls and in particular optical brighteners of the formulas:

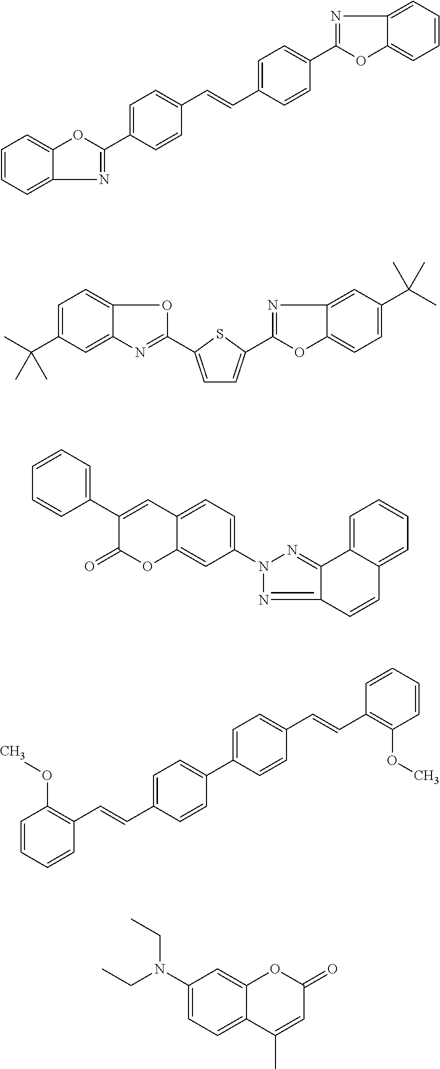

Suitable filler deactivators are, for example, polysiloxanes, polyacrylates, in particular block copolymers such as polymethacrylic acid polyalkyene oxide or polyglycidyl(meth)acrylates and their copolymers, e.g.

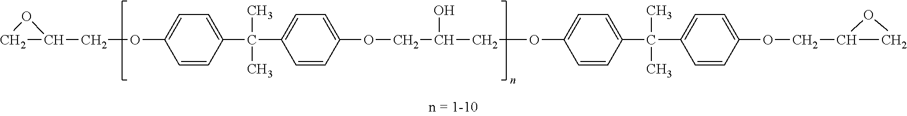

with styrene and epoxides of e.g. the following structures:

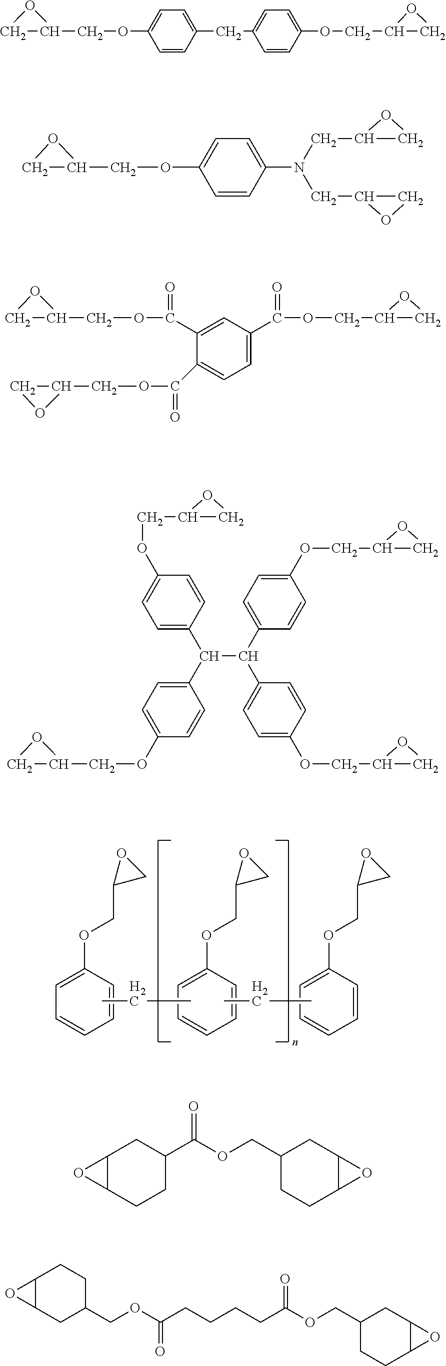

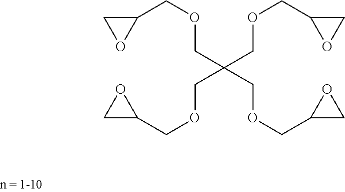 [SMILES: C(OCC(COCC1CO1)(COCC1CO1)COCC1CO1)C1CO1]

Suitable antistatic agents are, for example, ethoxylated alkylamines, fatty acid esters, alkylsulfonates, and polymers such as e.g. polyetheramides.

Suitable antiozonants are the above-named amines such as e.g. N,N′-di-isopropyl-p-phenylene diamine, N,N′-di-sec-butyl-p-phenylene diamine, N,N′-bis(1,4-dimethylpentyl)-p-phenylene diamine, N,N′-dicyclohexyl-p-phenylene diamine, N-isopropyl-N′-phenyl-p-phenylene diamine, N-(1,3-dimethylbutyl)-N′-phenyl-p-phenylene diamine, N-(1-methylheptyl)-N′-phenyl-p-phenylene diamine, N-cyclohexyl-N′-phenyl-p-phenylene diamine.

Suitable rheology modifications, e.g. for the preparation of controlled rheology polypropylene (CR-PP) are, for example, peroxides, alkoxyaminoesters or oxymide sulfonic acid esters.

Suitable additives for the linear molecular weight structure of polycondensation polymers (chain extenders) are diepoxides, bis-oxazonlines, bis-oxazolones, bis-oxazines, diisoscyanates, dianhydrides, bis-acyllactams, bis-maleimides, dicyanates, carbodiimides and polycarbodiimides. Further suitable chain extenders are polymeric compounds, such as e.g. polystyrene-polyacrylate-polyglycidyl (meth)acrylate copolymers, polystyrene-maleic anhydride copolymers and polyethylene-maleic anhydride copolymers.

Suitable hydrolysis stabilizers for polycondensation polymers, such as polyesters or polyamides, are, for example, epoxides, carbodiimides, polycarbodiimides or aziridines.

Suitable additives to increase the electrical conductivity are, for example, the mentioned static inhibitors, black carbon, and carbon compounds such as carbon nanotubes and graphene, metal powders such as e.g. copper powder, and conductive polymers such as e.g. polypyrroles, polyanilines, and polythiopenes.

Suitable infrared-active additives are, for example, aluminum silicates or dyestuffs such as phthalocyanines or anthraquinones.

Suitable crosslinking agents are, for example, peroxides like dialkyl peroxides, alkylaryl peroxides, peroxyesters, peroxycarbonates, diacylproxides, peroxyketals, silanes, such as e.g. vinyltrimethoxysilane, vinyltriethoxysilane, vinyltriacetoxysilane, vinyltris(2-methoxyethoxy)silane, 3-methacryloyloxypropyltrimethoxysilane, vinyldimethoxymethylsilane or ethylene-vinylsilane copolymers.

Suitable additives to increase the thermal conductivity of plastic recyclates are, for example, inorganic fillers such as boron nitride, aluminum nitride, aluminum oxide, aluminum silicate, silicon carbode, but also carbon nanotubes (CNT).

Suitable impact modifiers are usually selected for the particular polymer and are selected for example from the group of functionalized or non-functionalized polyolefins, such as e.g. ethylene copolymers such as EPDM or maleic anhydride or styrene-acrylonitrile-modified EPDM, glycidyl-methacrylate-modified ethylene-acrylate copolymers or also ionomers, core-shell polymers for example based on MBS (methacrylate-butadiene-styrene copolymer) or acrylester-polymethyl methacrylate, thermoplastic elastomers (TPE) for example based on styrene-block copolymers (styrene-butadiene (SB), styrene-butadiene-styrene (SBS) optionally hydrated (SEBS) or modified by maleic anhydride (SEBS-g-MAH), thermoplastic polyurethanes, copolyesters or copolyamides.

Suitable slip agents are, for example, amide waxes such as erucic acid amide or oleic acid amide.

Suitable antiblocking agents are, for example, silica, talc or zeolites.

Suitable demolding agents are, for example, silicones, soaps, and waxes, such as e.g. montan waxes.

In the above-named preferred embodiment, the at least one additive can be contained or added in an amount of 0.01 to 9.99% by weight, preferably 0.01 to 4.98% by weight, more preferably 0.02 to 2.00% by weight, particularly preferably 0.05 to 1.00% by weight, based on the total of the compound of Formula I, the organic material and the at least one additive.

In addition, an organic material is proposed in accordance with the invention, in particular a plastic composition, which contains the at least one compound of general Formula I, and/or at least one polymeric compound containing a repeating unit in accordance with general Formula II or a mixture of a plurality of compounds in accordance with general Formula I and/or polymeric compounds containing a repeating unit in accordance with general Formula II, as previously defined, as a stabilizer.

One preferred embodiment provides an organic material with the following composition:
0.01 to 10.00% by weight, preferably 0.02 to 5.00% by weight, more preferably 0.05 to 3.00% by weight, more preferably 0.10 to 2.00% by weight, particularly preferably 0.10 to 1.00% by weight of at least one compound of general Formula I, at least one polymeric compound containing a repeating unit according to general Formula II or a mixture of a plurality of the compounds according to general Formula I and/or polymeric compounds containing a repeating unit according to general Formula II,
99.99 to 90.00% by weight, preferably 99.89 to 95.00% by weight, more preferably 99.93 to 96.98% by weight, particularly preferably 99.90 to 98.00% by weight, of at least one organic material, preferably selected from the group consisting of plastics, coatings, lubricants, hydraulic oils, engine oils, turbine oils, transmission oils, metal machining fluids, chemicals, or monomers, and
0 to 9.99% by weight, preferably 0 to 4.98% by weight, particularly preferably 0.02 to 2.00% by weight of at least one additive,
wherein the components add up to 100% by weight.

The organic material can for example at least one additive, selected from the group consisting of secondary and/or primary antioxidants, in particular primary and/or secondary antioxidants selected from the group consisting of phosphites, phosphonites, thiols, phenolic antioxidants, sterically hindered amines, hydroxylamines, and mixtures or combinations thereof, UV absorbers, light stabilizers, stabilizers on a hydroxylamine base, stabilizers on a benzofuranone base, nucleating agents, toughness improvers, plasticizers, mold lubricants, rheological modifiers, chain extenders, processing aids, pigments, dyes, optical brighteners, antimicrobial active agents, antistatic agents, slip agents, anti-blocking agents, coupling agents, dispersing agents, compatibilizers, oxygen scavengers, acid scavengers, costabilizers, marking agents, and anti-fogging agents, in particular secondary antioxidants.

In particular, the at least one additive is thereby selected from the group consisting of a secondary antioxidant selected from the group consisting of phosphites, phosphonites, at least one costabilizer selected from the group consisting of polyols, acid scavengers, and sterically hindered amines.

The invention additionally relates to a method for stabilizing organic materials in particular against oxidative, thermal and/or actinic degradation, in which at least one compound of general Formula I, at least one polymeric compound containing a repeating unit in accordance with general Formula II or a mixture of a plurality of compounds in accordance with general Formula I and/or polymeric compounds containing a repeating unit in accordance with general Formula II, as previously defined, is incorporated into the organic material.

In accordance with the invention the following compounds are additionally proposed which are suitable as stabilizers:

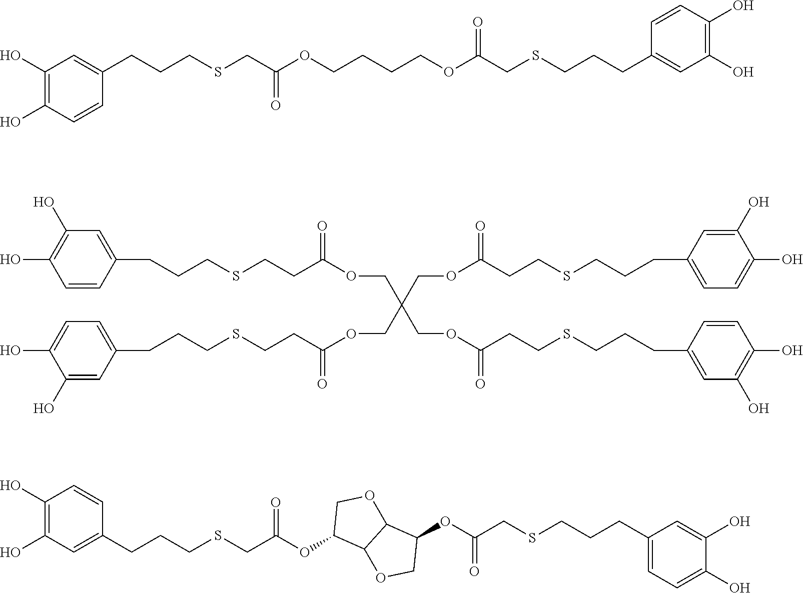 [SMILES: O=C(CSCCCc1ccc(O)c(O)c1)OCCCCOC(=O)CSCCCc1ccc(O)c(O)c1 ; O=C(CCSCCCc1ccc(O)c(O)c1)OCC(COC(=O)CCSCCCc1ccc(O)c(O)c1)(COC(=O)CCSCCCc1ccc(O)c(O)c1)COC(=O)CCSCCCc1ccc(O)c(O)c1 ; O=C(CSCCCc1ccc(O)c(O)c1)O[C@H]1COC2C1OC[C@H]2OC(=O)CSCCCc1ccc(O)c(O)c1]

or a polymeric compound containing a repeating unit according to general Formula II

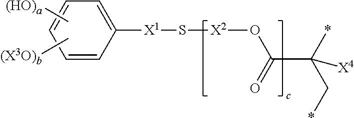

where X¹, X², X³, a, b and c are defined as previously.

In addition, a method is proposed for producing a compound of general Formula I, in which method for producing a compound of general Formula I according to any one of claims 1 to 3, in which
a compound of general Formula II

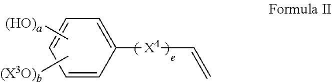

is reacted in a first step with a protective group reagent to provide a compound of general Formula IIIa or IIb,

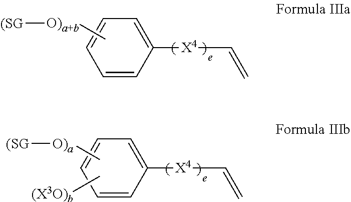

where the variables X², X³, a, b, c and d have the meanings specified above, and
X⁴ is a linear or branched alkylene radical having 1 to 15 carbon atoms, preferably a linear alkylene radical having 1 to 3,
SG is a protective group and
e is 0 or 1;
in a second step, the compound of general Formula IIIa or IIIb is reacted with, for example, 1/d equivalents of a compound of general Formula IV

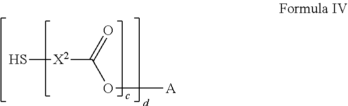

to produce the compound according to general Formula I, and

the product obtained in the second step is subsequently deprotected.

In the previously listed method, the reaction with 1/d equivalents means that preferably at least 1/d equivalents of the compound of general Formula IV is present for the reaction. However, it can also work with an excess of the compound of general Formulas III or IV relative to the other reaction partners.

The present invention will be described in greater detail by way of the subsequent examples without limiting the examples to preferred embodiments.

### EMBODIMENTS

### A) Production of Stabilizers in Accordance with the Invention

### a. Synthesis of the Triethylsilyl-Protected Eugenol

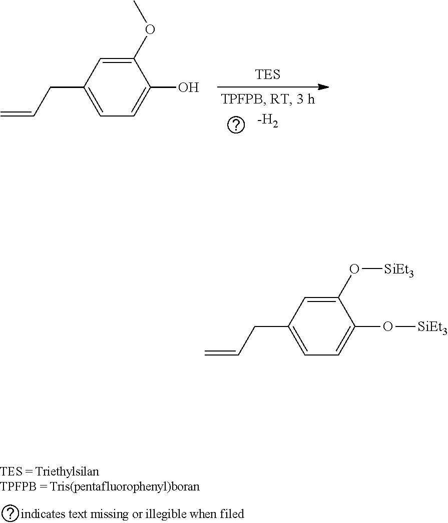

In a heated 250 ml three-necked flask with a septum, reflux condenser and a nitrogen inlet, 125 mg (1.00 eq., 0.24 mmol) of tris(pentafluorophenyl)borane are first placed in a nitrogen countercurrent. In a separate 100 mL Schlenk flask, 42.00 mL (2.02 eq, 30.66 g, 263.67 mmol) triethylsilane and 20 mL (1.00 eq, 21.40 g, 130.33 mmol) eugenol are stirred together for 10 min. The triethylsilane-eugenol mixture is then slowly added to the three-necked flask via a septum using a nitrogen-purged syringe, wherein a strong formation of gas and generation of heat occurs. The reaction mixture subsequently turns a yellow color which, however, disappears again during the course of stirring at room temperature for 4 hours. After the reaction time has elapsed, the reaction mixture is taken up in 100 mL dichloromethane and passed through a neutral aluminum oxide column. The reaction mixture is concentrated by rotary evaporation and residues of triethylsilane still present are distilled off under vacuum. 46.93 g of a light yellow liquid are obtained. The yield amounts to 95.20%.

### b. Synthesis of the Triethylsilyl-Protected 1,4-Butanediol Bis(Thioglycolate) Urushiol Thioether

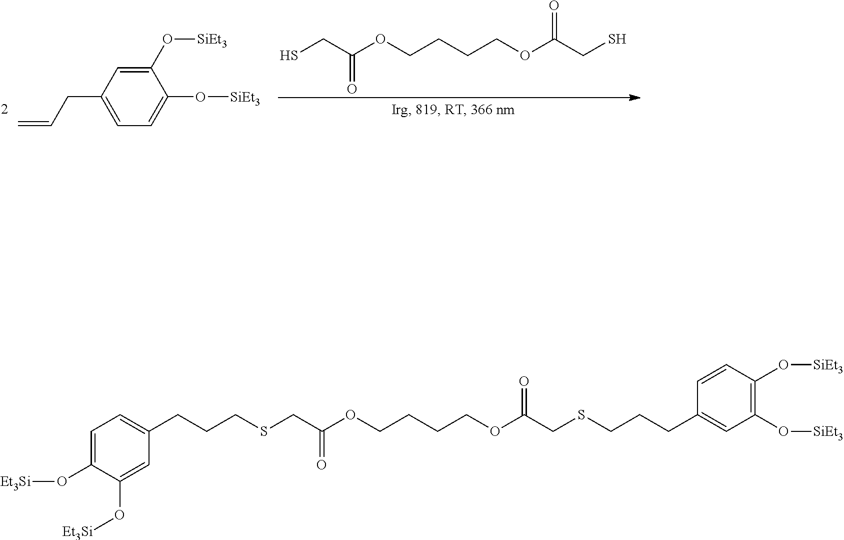

In a 100 mL Schlenk flask, 2.20 g (1.00 eq., 9.23 mmol) 1,4-butanediol bis(thioglycolate) are mixed with 7.00 g (2.00 eq., 18.51 mmol) of the triethyl-protected eugenol. The reaction mixture is degassed once using the freeze-pump-thaw method and then a small amount of Irgacure 819 is added in a nitrogen countercurrent. Afterwards, the reaction mixture is irradiated with stirring at a wavelength of 366 nm under a nitrogen atmosphere. The progress of the reaction is followed by means of ¹H-NMR spectroscopy by taking regular samples. A significant increase in viscosity can already be seen after 30 min and the reaction is complete. after 20 h. The yield amounts to 99.50%.

### c. Deprotection of the Triethylsilyl-Protected 1,4-Butanediol Bis(Thioglycolate) Urushiol Thioether

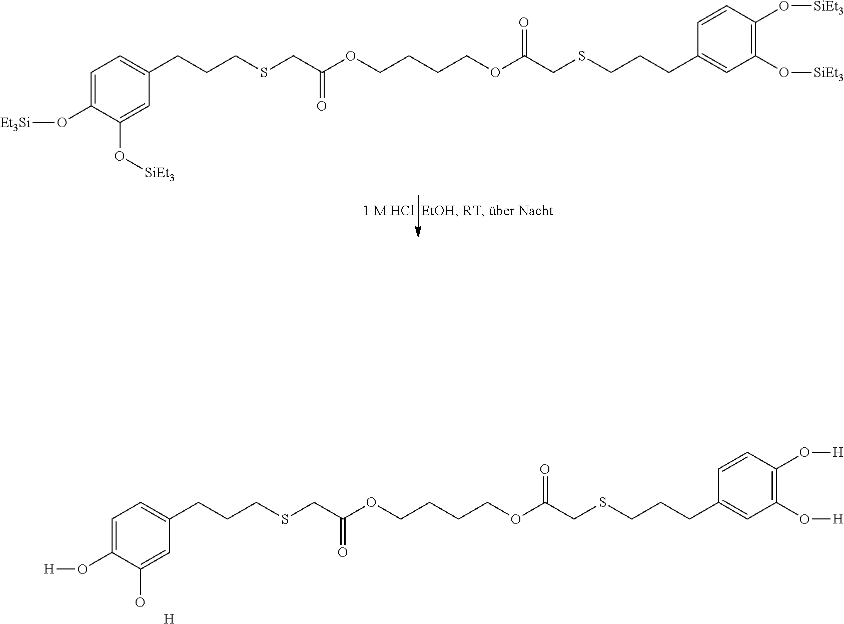

In a 100 mL round bottom flask, 3.00 g (3.02 mmol) of the triethylsilane-protected 1,4-butanediol bis(thioglycolate) urushiol thioether are dissolved in 30 mL of ethanol. 1.20 mL of 1 M hydrochloric acid are then added, wherein a white turbidity occurs, which subsequently disappears again. The reaction mixture is stirred overnight and 15 mL of a saturated sodium bicarbonate solution and 40 mL of distilled water are added the following day. The reaction mixture is extracted three times with 30 mL ethyl acetate each time. The combined organic extracts are washed again with 40 mL of a saturated sodium chloride solution and finally evaporated. The residue is taken up in 60 mL tetrahydrofuran and passed through a frit with a thin layer of silica gel. The filtrate is concentrated by rotary evaporation and finally distilled again under vacuum. After cooling, 1.31 g (2.43 mmol) of a white, waxy solid are obtained. The yield amounts to 80.46%.

TABLE 1
Overview of the thermogravimetric analyses of the synthesized
1,4-butanediol bis(thioglycolate) urushiol thioether.

| Loss of mass [%] | Temperature [° C.] |
|---|---|
| 5.0 | 289.81 |
| 10.0 | 311.44 |
| 50.0 | 367.33 |

### d. Synthesis of the Triethylsilyl-Protected Pentaerythritol Tetrakis(3-Mercaptopropionate) Urushiol Thioether

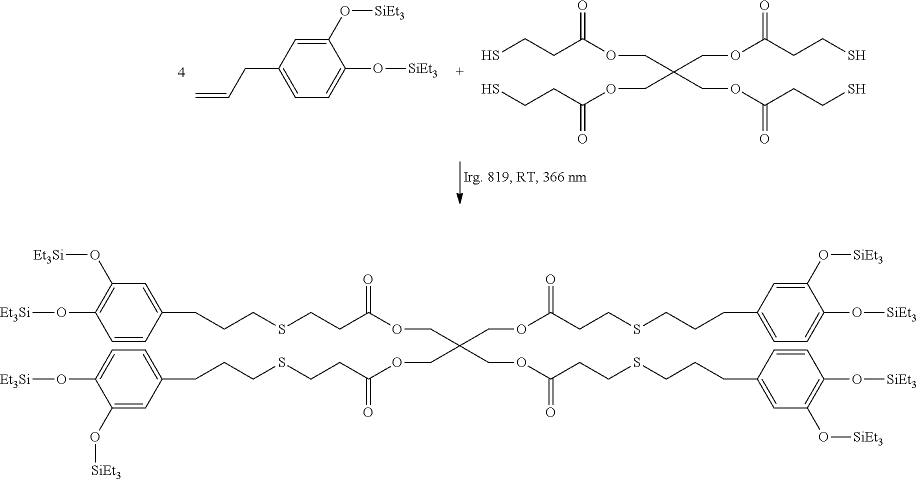

In a 100 mL Schlenk flask, 6.40 g (1.00 eq., 13.10 mmol) pentaerythritol tetrakis(3-mercaptopropionate) are mixed with 19.82 g (4.00 eq., 52.40 mmol) of the triethyl-protected eugenol. The reaction mixture is degassed once using the freeze-pump-thaw method and then a small amount of IRGACURE 819 is added in a nitrogen countercurrent. Afterwards, the reaction mixture is irradiated with stirring at a wavelength of 366 nm under a nitrogen atmosphere. The progress of the reaction is followed by means of ¹H-NMR spectroscopy by taking regular samples. A significant increase in viscosity can already be seen after 30 min and the reaction is complete. after 48 h. The yield amounts to 99.87%.

### e. Deprotection of the Triethylsilyl-Protected Pentaerythritol Tetrakis(3-Mercaptopropionate) Urushiol Thioether

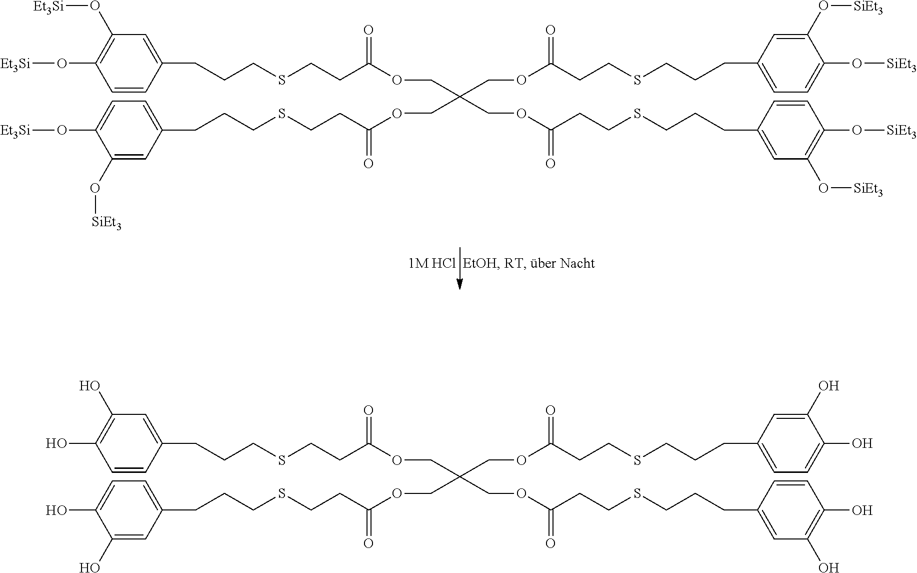

In a 100 mL round bottom flask, 3.02 g (1.51 mmol) of the triethylsilane-protected pentaerythritol tetrakis(3-mercaptopropionate) urushiol thioether are placed in 30 mL ethanol. 1.50 mL of 1 M hydrochloric acid are then added, wherein a white turbidity occurs, which subsequently disappears again. The reaction mixture is stirred overnight and 15 mL of a saturated sodium bicarbonate solution and 40 mL of distilled water are added the following day. The reaction mixture is extracted three times with 30 mL ethyl acetate each time. The combined organic extracts are washed again with 40 mL of a saturated sodium chloride solution and finally evaporated. The residue is taken up in 60 mL tetrahydrofuran and passed through a frit with a thin layer of silica gel. The filtrate is concentrated by rotary evaporation and finally distilled again under vacuum. After cooling, 1.09 g (1.00 mmol) of a light yellow, viscous liquid are obtained. The yield amounts to 66.23%.

TABLE 2
Overview of the thermogravimetric analyses
of the synthesized pentaerythritol tetrakis(3-
mercaptopropionate) urushiol thioether.

| Loss of mass [%] | Temperature [° C.] |
|---|---|
| 5.0 | 282.30 |
| 10.0 | 319.71 |
| 50.0 | 379.18 |

### f. Synthesis of the Triethylsilyl Octadecanethiol Urushiol Thioether

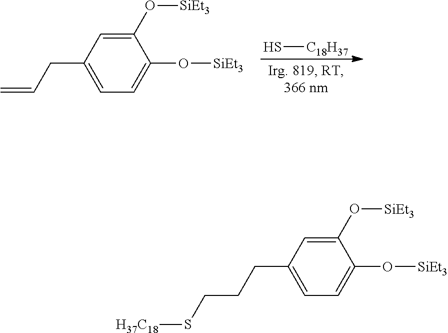

In a 100 mL Schlenk flask, 5.48 g (1.00 eq., 19.12 mmol) octadecanethiol are mixed with 7.00 g (0.97 eq., 18.51 mmol) of the triethyl-protected eugenol. The reaction mixture is degassed once using the freeze-pump-thaw method and then a small amount of IRGACURE 819 is added in a nitrogen countercurrent. Afterwards, the reaction mixture is irradiated with stirring at a wavelength of 366 nm under a nitrogen atmosphere. The progress of the reaction is followed by means of ¹H-NMR spectroscopy by taking regular samples. A significant increase in viscosity can already be seen after 30 min and the reaction is complete. after 24 h. The yield amounts to 99.27%.

### g. Deprotection of the Triethylsilyl Octadecanethiol Urushiol Thioether

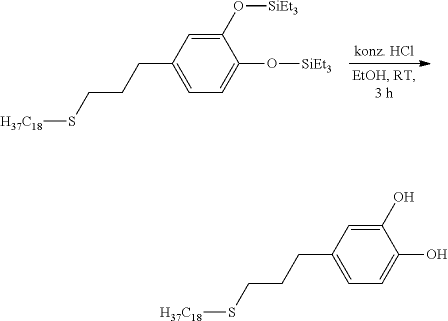

In a 100 mL round bottom flask, 5.40 g (8.13 mmol) of the triethylsilane-protected octadecanethiol urushiol thioether are dissolved in 30 mL of ethanol. 0.8 mL of concentrated hydrochloric acid are then added, wherein a white turbidity occurs, which subsequently disappears again After 3 h, 10 mL of distilled water are added dropwise to the solution, wherein a white precipitate separates out. This is filtered off and finally recrystallized from methanol. 3.36 g (7.70 mmol) of a white solid are obtained. The yield amounts to 94.72%.

TABLE 3
Overview of the thermogravimetric analyses of the
synthesized octadecanethiol urushiol thioether.

| Loss of mass [%] | Temperature [° C.] |
|---|---|
| 5.0 | 295.80 |
| 10.0 | 318.62 |
| 50.0 | 373.76 |

### h. Synthesis of (3R,6S)-hexahydrofuro[3,2-b]furan-3,6-diyl bis(3-((3-(3,4-bis((triethylsilyl)oxy)phenyl)propyl)thio)propanoate)

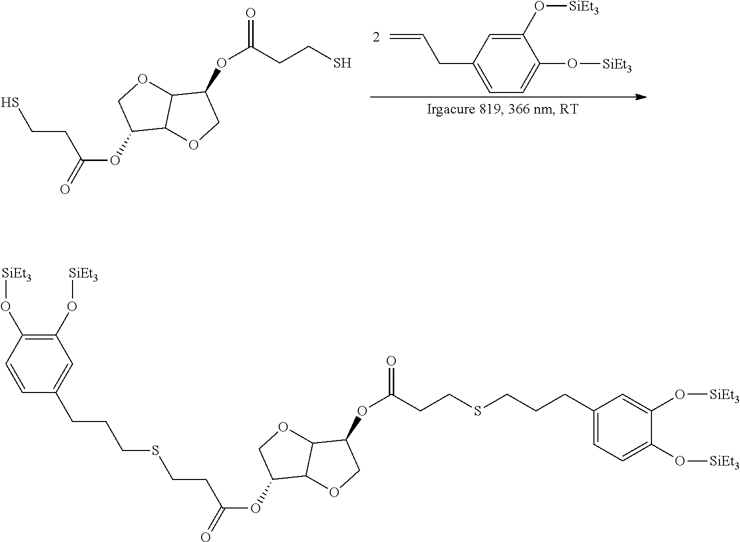

In a 100 mL Schlenk flask, 7.00 g (1.00 eq., 21.74 mmol) isosorbide bis-(3-mercapto)propionate are placed in 16.44 g (2.00 eq., 43.47 mmol) of the triethyl-protected eugenol. The reaction mixture is degassed once using the freeze-pump-thaw method and then a small amount of IRGACURE 819 is added in a nitrogen countercurrent. Afterwards, the reaction mixture is irradiated with stirring at a wavelength of 366 nm under a nitrogen atmosphere, wherein the reaction mixture is gradually homogenized. The progress of the reaction is followed by means of ¹H-NMR spectroscopy by taking regular samples. The reaction mixture has homogenized after 24 h and the reaction is complete after 96 h. The yield amounts to 99.26%.

### i. Deprotection of (3R,6S)-hexahydrofuro[3,2-b]furan-3,6-diyl bis(3-((3-(3,4-bis((triethylsilyl)oxy)phenyl)propyl)thio)propanoate)

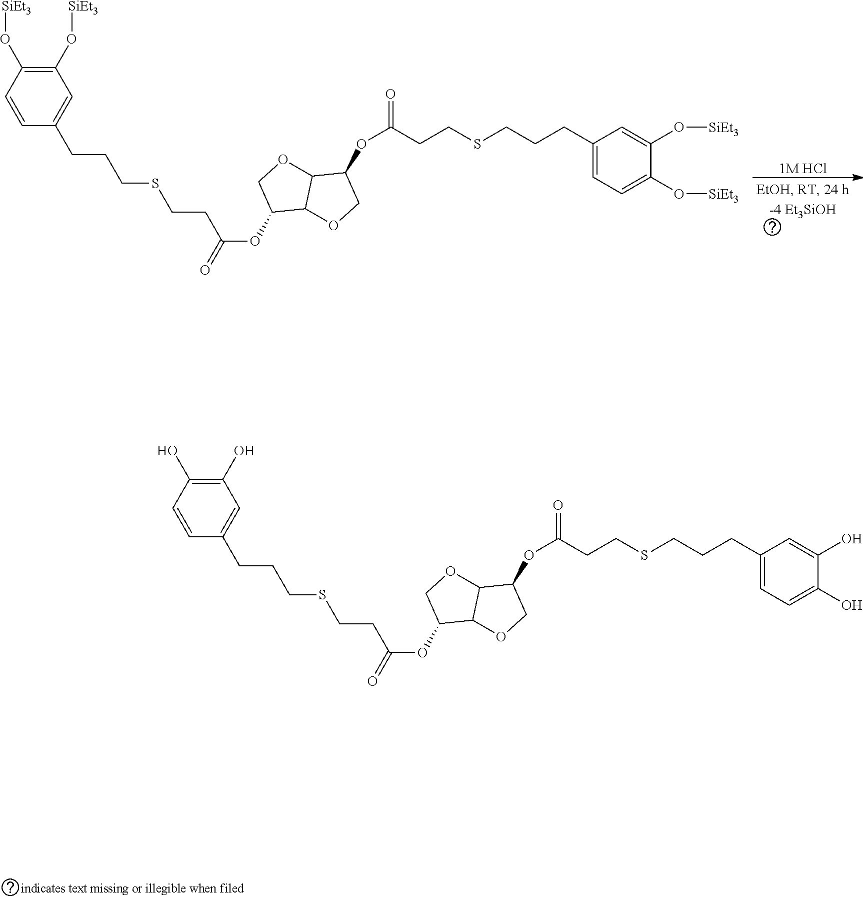

7.32 g (1.00 eq., 6.79 mmol) (3R,6S)-hexahydrofuro[3,2-b]furan-3,6-diyl bis(3-((3-(3,4-bis((triethylsilyl)oxy)phenyl)propyl)thio)propanoate) and 35 mL ethanol are placed in a 100 mL round bottom flask. 3.5 mL of 1M hydrochloric acid are added while stirring, wherein in a white, voluminous precipitate results, which redissolves. After 24 h, the reaction mixture is poured into 40 mL saturated sodium bicarbonate and the aqueous solution is extracted three times with 40 mL ethyl acetate each time. The organic phase is dried over sodium sulfate and the solvent is then removed on a rotary evaporator. Finally, the residue is distilled again under high vacuum, wherein 2.73 g (4.38 mmol) of a highly viscous, red-orange gel remains. The yield amounts to 64.51%.

TABLE 4
Overview of the results of the thermogravimetric
analysis of hexahydrofuro[3,2-b]furan-3,6-
diyl bis((3-((3-(3,4-dihydroxyphenyl)propyl)thio)propanoate
under nitrogen atmosphere.

| Loss of mass [%] | Temperature [° C.] |
|---|---|
| 5.0 | 278.12 |
| 10.0 | 299.22 |
| 50.0 | 351.33 |

### j. Thiol-ene reaction of mercaptoethanol with the triethylsilyl-protected eugenol

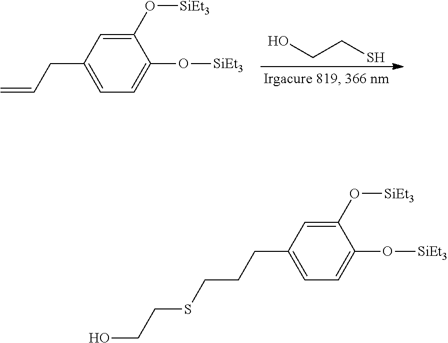

3.70 mL (1.00 eq., 53.03 mmol) mercaptoethanol and 20.02 g (1.00 eq., 52.93 mmol) of the triethyl-protected eugenol are placed in a 100 mL Schlenk flask. The reaction mixture is degassed once using the freeze-pump-thaw method and then a small amount of Irgacure 819 is added in a nitrogen countercurrent. Afterwards, the reaction mixture is irradiated with stirring at a wavelength of 366 nm under a nitrogen atmosphere. The progress of the reaction is followed by means of ¹H-NMR spectroscopy by taking regular samples. A significant increase in viscosity can already be seen after 15 min and the reaction is complete. after 12 hours. Excess mercaptoethanol is removed by vacuum distillation. The yield amounts to 99.87%.

### k. Methacrylate of Hydroxy-End-Functionalized Triethylsilyl-Protected Eugenol

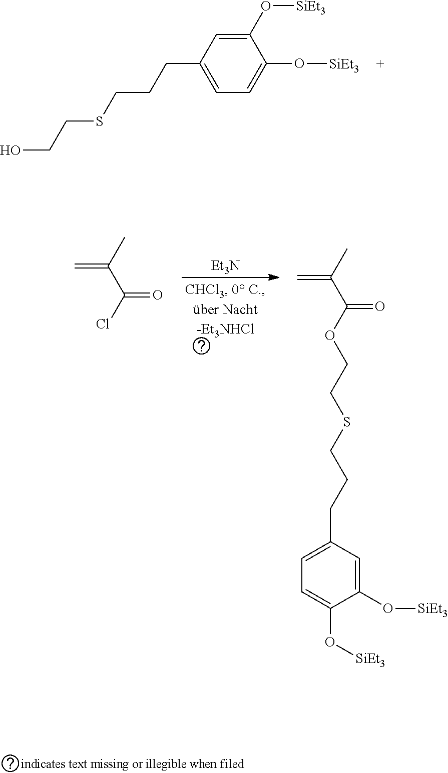

In a heated 500 mL three-necked flask with dropping funnel and septum, 22.03 g (1.00 eq., 48.28 mmol) of the hydroxy-end-functionalized triethylsilyl-protected eugenol are dissolved via the thiol-ene reaction with mercaptoethanol in 100 mL dried chloroform in a nitrogen atmosphere. 10.10 mL (1.51 eq., 72.86 mmol) triethylamine are then added in a nitrogen countercurrent. The reaction mixture is cooled for 30 min in an ice bath. Afterwards, a solution of 5.10 mL (1.1 eq., 53.03 mmol) methacryloyl chloride and 60 mL dry chloroform is slowly added dropwise. After the addition is complete, the reaction mixture is stirred at room temperature overnight and washed 3 times with distilled water the following day. The reaction mixture is then passed through a neutral aluminum oxide column and the solvent is evaporated by rotary evaporation. After cooling, 13.54 g (25.79 mmol) of a yellow, viscous liquid are obtained. The yield amounts to 53.43%.

### l. Synthesis of poly(2-((3-(3,4-dihydroxyphenyl)propyl)thio)ethyl methacrylate)

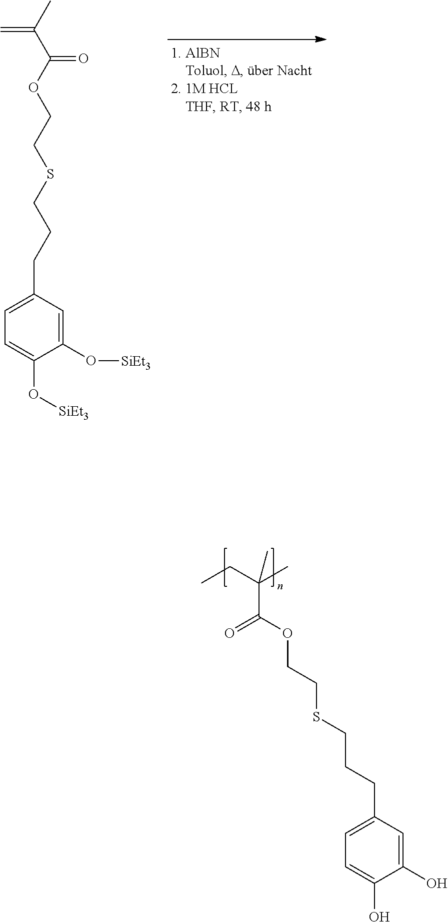

In a 100 mL Schlenk flask, 10.00 g (1.00 eq., 19.07 mmol) of 2-((3-(3,4-dihydroxyphenyl)propyl)thio)ethyl methacrylate and 90 mg (0.03 eq., 0.55 mmol) azobis(isobutyronitrile) recrystallized from methanol are dissolved in 40 mL toluene. The solution is degassed three times using the freeze-pump-thaw method and the reaction solution is then heated to 73° C. overnight under a nitrogen atmosphere. A significant increase in viscosity can be seen after 30 min. The flask is immediately transferred to an ice bath the following day and the polymer is then precipitated in 300 mL of methanol. After drying, the transparent gel is taken up in 100 mL tetrahydrofuran and mixed with 3.00 mL 1 M hydrochloric acid. After 48 h, the polymer is then precipitated in 400 mL of n-hexane. After drying at 80° C. under high vacuum, 3.57 g of a white-beige solid are obtained.

TABLE 5
Overview of the results of the thermogravimetric analysis
of the poly(2-((3-(3,4-dihydroxyphenyl)propyl)thio)ethyl
methacrylate) under nitrogen atmosphere.

| Loss of mass [%] | Temperature [° C.] |
|---|---|
| 5.0 | 283.69 |
| 10.0 | 305.23 |
| 50.0 | 358.09 |

TABLE 6
Overview of the average molecular weight
and the dispersity of the synthesized
poly(2-((3-(3,4-dihydroxyphenyl)propyl)thio)ethyl
methacrylate) determined by means of gel
permeation chromatography.

| Mn [g/mol] | Ð |
|---|---|
| 11100 | 2.54 |

### m. Synthesis of Polystearyl methacrylate-co-poly(2-((3-(3,4-dihydroxyphenyl)propyl)thio)ethyl methacrylate) Copolymer

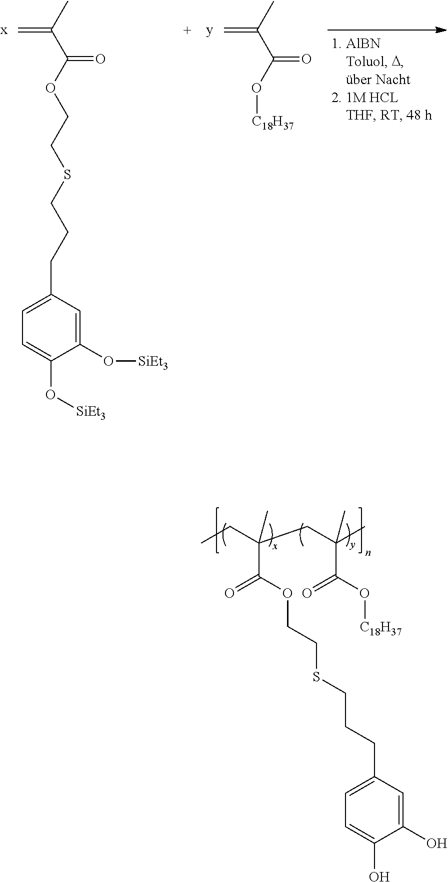

In a 100 mL Schlenk flask, 11.00 g (1.00 eq., 20.96 mmol) of 2-((3-(3,4-dihydroxyphenyl)propyl)thio)ethyl methacrylate, 3.05 g (0.43 eq., 9.01 mmol) of destabilized stearyl methacrylate and 72 mg (0.02 eq., 0.44 mmol) azobis(isobutyronitrile) recrystallized from methanol are dissolved in 32 mL toluene. The solution is degassed three times using the freeze-pump-thaw method and the reaction solution is then heated to 73° C. overnight under a nitrogen atmosphere. A significant increase in viscosity can be seen after 30 min. The flask is immediately transferred to an ice bath the following day and the polymer is then precipitated in 350 mL of methanol. After drying, the transparent gel is taken up in 100 mL tetrahydrofuran and mixed with 3.00 mL 1 M hydrochloric acid and a few drops of ethanol. The progress of the desilylation is checked by taking precipitation samples in n-hexane with subsequent ¹H-NMR analysis. After 360 h, the polymer is then precipitated in 500 mL of n-hexane. After drying at 80° C. under high vacuum, 3.08 g of a white-beige, slightly greasy solid are obtained.

TABLE 7
Overview of the results of the thermogravimetric
analysis of the polystearyl methacrylate-co-poly(2-
((3-(3,4-dihydroxyphenyl)propyl)thio)ethyl methacrylate)
copolymer under nitrogen atmosphere.

| Loss of mass [%] | Temperature [° C.] |
|---|---|
| 5.0 | 309.15 |
| 10.0 | 324.29 |
| 50.0 | 376.25 |

TABLE 8
Overview of the average molecular weight and the dispersity
of the synthesized random copolymer determined by
means of gel permeation chromatography.

| Mn [g/mol] | Ð |
|---|---|
| 29400 | 2.56 |

### B) Application Check

To examine the effect of the stabilizers according to the invention, a commercial polypropylene (Moplen HP 501N, Lyondell Basell Industries) was homogenized in a powder-powder mixture with the stabilizers stated in Table 9 and was circulated in a twin-screw microextruder (MC 5, manufacturer DSM) for 30 minutes at 200° C. and 200 revolutions per minute and the decrease in the force was recorded. The force is a direct measure for the molecular weight of polypropylene; the smaller the reduction, the higher the stabilization effect.

TABLE 9
Stabilization of polypropylene with antioxidants according to the invention

| | | Residual strength |
|---|---|---|
| | | after 10/20/30 |
| | | minutes [%], |
| | | respective mean |
| | | values from 2 tests) |
| | Additive (% by weight) | 0 min = 100% |
| Comparison example 1 | Without additive | 51/26/13 |
| Comparison example 2 | 0.5% pentaerythritol tetrakis(3-(3,5-di- | 84/79/75 |
| (commercial stabilizer) | tert-butyl-4-hydroxyphenyl)propionate) | |
| Comparison example 3 | 0.5% 5-octadecyl-3-(3,5-di-tert-butyl-4- | 82/71/60 |
| (commercial stabilizer) | hydroxyphenyl)propionate | |
| Example 1 in | 0.5% 1,4-butanediol bis(thioglycolate) | 97/95/94 |
| accordance with the | urushiol thioether | |
| invention | | |
| Example 2 in | 0.5% pentaerythritol tetrakis(3- | 96/96/95 |
| accordance with the | mercaptopropionate) urushiol thioether | |
| invention | | |
| Example 3 in | 0.5% octadecanethiol urushiol thioether | 96/94/91 |
| accordance with the | | |
| invention | | |
| Example 4 in | 0.5% hexahydrofuro[3,2-b]furan-3,6-diyl | 92/90/89 |
| accordance with the | bis((3-((3-(3,4- | |
| invention | dihydroxyphenyl)propyl)thio)propanoate | |
| Example 5 in | 0.5% poly(2-((3-(3,4- | 86/80/74 |
| accordance with the | dihydroxyphenyl)propyl)thio)ethyl | |
| invention | methacrylate) | |
| Example 6 in | 0.5% polystearyl methacrylate-co- | 90/85/81 |
| accordance with the | poly(2-((3-(3,4- | |
| invention | dihydroxyphenyl)propyl)thio)ethyl | |
| | methacrylate) | |

The additives in accordance with the invention show a very good stabilization effect since a smaller reduction of the polymer takes place over the trial period in comparison to an unstabilized polymer and a polymer stabilized with standard antioxidants.

In further experiments, stabilizer compositions according to the invention were tested with regard to their effect (Table 10).

TABLE 10
Stabilization of polypropylene with stabilizer
compositions according to the invention

| | | Residual strength |
|---|---|---|
| | | after 10/20/30 |
| | | minutes [%], |
| | | respective mean |
| | | values from 2 tests) |
| | Additive (% by weight) | 0 min = 100% |
| Comparison | Without additive | 51/26/13 |
| example 1 | | |
| Example 7 in | 0.25% 1,4-butanediol bis(thioglycolate) | 97/96/96 |
| accordance with | urushiol thioether | |
| the invention | 0.25% tris-(2,4-di-tert- | |
| | butylphenyl)phosphite | |
| Example 8 in | 0.25% pentaerythritol tetrakis(3- | 97/96/95 |
| accordance with | mercaptopropionate) urushiol thioether | |
| the invention | 0.25% tris-(2,4-di-tert- | |
| | butylphenyl)phosphite | |
| Example 9 | 0.25% octadecanethiol urushiol thioether | 97/95/94 |
| according to the | 0.25% tris-(2,4-di-tert- | |
| invention | butylphenyl)phosphite | |
| Example 10 | 0.25% hexahydrofuro[3,2-b]furan-3,6-diyl | 97/96/95 |
| according to the | bis((3-((3-(3,4- | |
| invention | dihydroxyphenyl)propyl)thio)propanoate | |
| | 0.25% tris-(2,4-di-tert- | |
| | butylphenyl)phosphite | |
| Example 11 | 0.25% 1,4-butanediol bis(thioglycolate) | 96/95/92 |
| according to the | urushiol thioether | |
| invention | 0,25% erythritol | |
| Example 12 | 0.25% poly(2-((3-(3,4- | 93/88/81 |
| according to the | dihydroxyphenyl)propyl)thio)ethyl | |
| invention | methacrylate) | |
| | 0.25% tris-(2,4-di-tert- | |
| | butylphenyl)phosphite | |
| Example 13 | 0.25% polystearyl methacrylate-co-poly(2- | 92/87/83 |
| according to the | ((3-(3,4-dihydroxyphenyl)propyl)thio)ethyl | |
| invention | methacrylate) | |
| | 0.25% tris-(2,4-di-tert- | |
| | butylphenyl)phosphite | |

The compositions in accordance with the invention display a very good stabilization effect since a smaller reduction of the polymer takes place over the trial period in comparison to the comparison examples.

### C) Oxidation Induction Time Analyses

The determination of the oxidation induction time (OIT) represents a possible method for assessing the efficacy of stabilizers. This analysis method is based on the reaction of the polymer to be examined with atmospheric oxygen. The sample is thereby initially melted and equilibrated under an inert gas atmosphere up to the selected measuring temperature above the melting temperature of the polymer. A switch of the flushing gas from inert gas to air is then carried out, wherein the heat flow is detected over the course of time. During consumption of the added stabilizer, an increase in the heat flow occurs as a result of the exothermic thermo-oxidative damage to the polymer. The OIT value results from the determination of the time until oxidation occurs, that is, the onset. In principle, the higher the OIT value, the longer the sample was stabilized by the antioxidant and the effectiveness is correspondingly greater. Table 11 summarizes the OIT values for compounds in which the stabilizers described in the patent have been incorporated at 0.5% by weight, for various temperatures. Polypropylene (Moplen HP 500N, Lyondell Basell Industries) was used as the polymer.

TABLE 11
Overview of OIT values at 220° C. for stabilizer
compositions according to the invention.

| | | OIT value |
|---|---|---|
| | Additive (% by weight) | [min] |
| Comparison example 1 | Without additive | 5 |
| Comparison example 2 | 0.5% octadecyl-3-(3,5-di-tert-butyl-4- | 123 |
| (commercial stabilizer) | hydroxyphenyl)propionate (Irganox | |
| | 1076) | |
| Comparison example 3 | 0.5% 2-methyl-4,6- | 108 |
| (commercial stabilizer) | bis(octylsulfanylmethyl)phenol (Irganox | |
| | 1520) | |
| Example 1 in | 0.5% 1,4-butanediol bis(thioglycolate) | 82 |
| accordance with | urushiol thioether | |
| the invention | | |
| Example 2 in | 0.5% pentaerythritol tetrakis(3- | 70 |
| accordance with | mercaptopropionate) urushiol thioether | |
| the invention | | |
| Example 3 in | 0.5% hexahydrofuro[3,2-b]furan-3,6-diyl | 105 |
| accordance with | bis((3-((3-(3,4- | |
| the invention | dihydroxyphenyl)propyl)thio)propanoate | |
| Example 4 in | 0.5% octadecanethiol urushiol | 198 |
| accordance with | thioether | |
| the invention | | |
| Example 5 in | 0.5% poly(2-((3-(3,4- | 97 |
| accordance with | dihydroxyphenyl)propyl)thio)ethyl | |
| the invention | methacrylate) | |
| Example 6 in | 0.5% polystearyl methacrylate-co-poly(2- | 87 |
| accordance with | ((3-(3,4- | |
| the invention | dihydroxyphenyl)propyl)thio)ethyl | |
| | methacrylate) | |

All synthesized and described compounds lead to a substantial increase in the OIT value and thus contribute to a substantial increase in the thermo-oxidative stability of the polymer. The OIT value, in particular for the octadecanethiol urushiol thioether, lies considerably above the values for the compounds with the commercial stabilizers.

1-15. (canceled)

16. A method of stabilizing an organic material against oxidative, thermal and/or actinic degradation comprising combining the organic material with:
a compound according to general Formula I 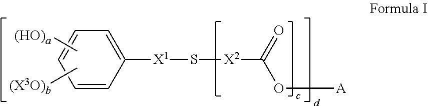
wherein, respectively independent of one another,
X¹ is a linear or branched alkylene radical having 2 to 18 carbon atoms,
X² is a linear or branched alkylene radical having 1 to 18 carbon atoms,
X³ is a linear or branched alkyl radical having 1 to 18 carbon atoms,
A is a d-valent saturated or unsaturated group,
a is 2 to 5-b,
b is 0 to 3,
c is 0 or 1, and
d is 1 to 8,
or
a polymeric compound containing a repeating unit according to general Formula II 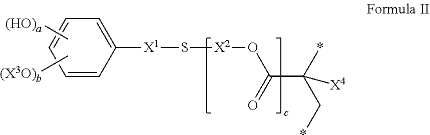
wherein
X¹, X², X³, a, b and c are defined as previously, and
X⁴ is hydrogen or a linear or branched alkyl radical having 1 to 18 carbon atoms, and
* is an attachment site of the repeating unit according to general Formula II,
or a mixture of a plurality of the compounds according to general Formula I and/or polymeric compounds containing a repeating unit according to general Formula II.

17. The method of claim 16, wherein
X¹ is a linear alkylene radical having 2 to 6,
X² is a linear alkylene radical having 1 to 6,
X³ is a linear or branched alkyl radical having 1 to 4 carbon atoms,
X⁴ is hydrogen or a linear or branched alkyl radical having 1 to 4 carbon atoms,
A is a d-valent, aliphatic or aromatic group,
a is 2, and
b is 0.

18. The method of claim 16, wherein the compound of general Formula I is one of the following compounds:

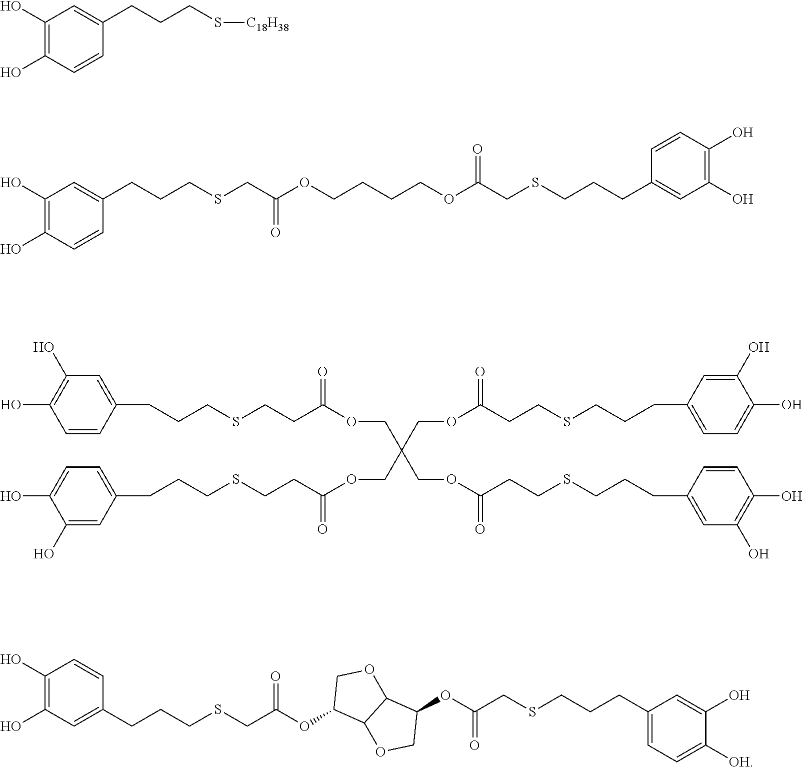

19. The method of claim 16, wherein the polymeric compound containing the repeating unit according to general Formula II is selected from the group consisting of homopolymers formed from repeating units according to general Formula II, or copolymers containing the repeating unit according to general Formula II and at least one further repeating unit derived from a radical polymerizable compound.

20. The method of claim 16, wherein the repeating unit according to general Formula II has the following structure

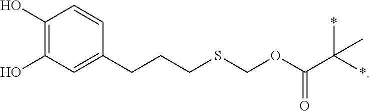

21. The method of claim 16, wherein the organic material is one or more of plastics, coatings, lubricants, hydraulic oils, engine oils, turbine oils, transmission oils, metal machining fluids, chemicals, and monomers.

22. The method of claim 16, wherein all of the compounds according to general Formula I, the polymeric compound containing a repeating unit according to general Formula II or the mixture of a plurality of the compounds according to general Formula I, and/or the polymeric compounds containing a repeating unit according to general Formula II are contained in the organic material at a proportion by weight of 0.01 to 10.00 by weight.

23. The method of claim 16, wherein the plastic is selected from the group consisting of
a) polymers made from olefins or diolefins, polyalkylene-carbon monoxide copolymers, and copolymers in the form of random or block structures,
b) polystyrene, polymethylstyrene, poly-alpha-methylstyrene, polyvinylnaphthalene, polyvinylbiphenyl, polyvinyltoluene, styrene-butadiene (SB), styrene-butadiene-styrene (SBS), styrene-ethylene-butylene-styrene (SEBS), styrene-ethylene-propylene-styrene, styrene-isoprene, styrene-isoprene-styrene (SIS), styrene-butadiene-acrylonitrile (ABS), styrene-acrylonitrile (SAN), styrene-acrylonitrile-acrylate (ASA), styrene-ethylene, styrene-maleic anhydride polymers including corresponding graft copolymers, graft copolymers made of methyl methacrylate, styrene-butadiene and ABS (MABS), and hydrogenated polystyrene derivatives,
c) halogen-comprising polymers,
d) polymers of unsaturated esters,
e) polymers of unsaturated alcohols and derivatives,
f) polyacetals, copolymers, and blends thereof
g) polymers of cyclic ethers,
h) polyphenylene oxides and blends thereof with polystyrene and/or polyamides, polyurethanes and polyureas,
j) polyamides and blends of polyamides and polyolefins,
k) polyimides, polyamide-imides, polyetherimides, polyesterimides, poly(ether)ketones, polysulfones, polyethersulfones, polyarylsulfones, polyphenylene sulfides, polybenzimidazoles, and polyhydantoins,
l) polyesters made from aliphatic or aromatic dicarboxylic acids and diols or from hydroxycarboxylic acids,
m) polycarbonates, polyester carbonates, and blends,
n) cellulose derivatives,
o) epoxy resins,
p) phenol resins,
q) unsaturated polyester resins produced from unsaturated dicarboxylic acids and diols with vinyl compounds, and alkyd resins,
r) silicones and silicones terminated with vinyl groups, and
s) mixtures, combinations, or blends of two or more of the above-named polymers.

24. The method of claim 16, wherein at least one further additive is combined or added, wherein the further additive is selected from the group comprising primary antioxidants, secondary antioxidants, UV absorbers, light stabilizers, metal deactivators, filler deactivators, antiozonants, nucleation agents, anti-nucleation agents, toughening agents, plasticizers, lubricants, rheological modifiers, thixotropic agents, chain extenders, optical brighteners, antimicrobial active agents, antistatic agents, slip agents, anti-blocking agents, coupling agents, crosslinking agents, branching agents, anti-cross-linking agents, hydrophilization agents, hydrophobing agents, bonding agents, dispersing agents, compatibilizers, oxygen scavengers, acid scavengers, expanding agents, degradation additives, defoaming agents, odor scavengers, marking agents, anti-fogging agents, additives to increase the electrical conductivity and/or thermal conductivity, infrared absorbers or infrared reflectors, gloss improvers, matting agents, repellents, fillers, reinforcement materials, and mixtures thereof.

25. The method of claim 16, wherein the at least one additive is combined or added in an amount of 0.01 to 9.99% by weight based on the total of the compound according to general Formula I, the organic material, and the at least one additive.

26. A composition comprising an organic material and at least one compound of general Formula I, at least one polymeric compound containing a repeating unit in accordance with general Formula II, or a mixture of a plurality of compounds in accordance with general Formula I and/or polymeric compounds containing a repeating unit in accordance with general Formula II, wherein the compound according to general Formula I is: 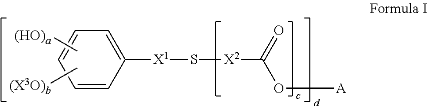
wherein, respectively independent of one another,
X¹ is a linear or branched alkylene radical having 2 to 18 carbon atoms,
X² is a linear or branched alkylene radical having 1 to 18 carbon atoms,
X³ is a linear or branched alkyl radical having 1 to 18 carbon atoms,
A is a d-valent saturated or unsaturated group,
a is 2 to 5-b,
b is 0 to 3,
c is 0 or 1, and
d is 1 to 8,
the polymeric compound containing a repeating unit according to general Formula II is 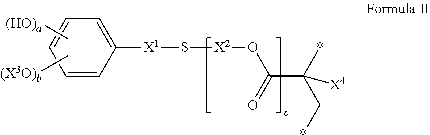
wherein
X¹, X², X³, a, b and c are defined above,
X⁴ is hydrogen or a linear or branched alkyl radical having 1 to 18 carbon atoms, and
* is an attachment site of the repeating unit according to general Formula II,
or a mixture of a plurality of the compounds according to general Formula I and/or polymeric compounds containing a repeating unit according to general Formula II.

27. The composition in accordance with claim 26, which has the following composition:
0.01 to 10.00% by weight of at least one compound of general Formula I, at least one polymeric compound containing a repeating unit according to general Formula II, or a mixture of a plurality of the compounds according to general Formula I and/or polymeric compounds containing a repeating unit according to general Formula II,
99.99 to 90.00% by weight of the at least one organic material, and
0 to 9.99% by weight of at least one additive,
wherein the components add up to 100% by weight.

28. The composition in accordance with claim 27, which is a plastic composition, and wherein the at least one additive is selected from the group consisting of secondary and/or primary antioxidants, UV absorbers, light stabilizers, hydroxylamine based stabilizers, benzofuranone based stabilizers, nucleating agents, toughness improvers, plasticizers, mold lubricants, rheological modifiers, chain extenders, processing aids, pigments, dyes, optical brighteners, antimicrobial active agents, antistatic agents, slip agents, anti-blocking agents, coupling agents, dispersing agents, compatibilizers, oxygen scavengers, acid scavengers, costabilizers, marking agents, and anti-fogging agents.

29. A compound of one of the following formulas: 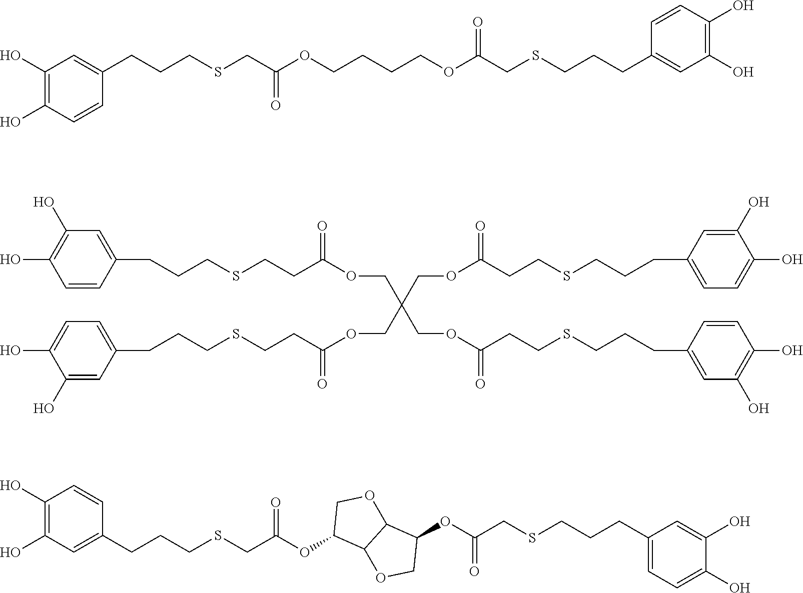 [SMILES: O=C(CSCCCc1ccc(O)c(O)c1)OCCCCOC(=O)CSCCCc1ccc(O)c(O)c1 ; O=C(CCSCCCc1ccc(O)c(O)c1)OCC(COC(=O)CCSCCCc1ccc(O)c(O)c1)(COC(=O)CCSCCCc1ccc(O)c(O)c1)COC(=O)CCSCCCc1ccc(O)c(O)c1 ; O=C(CSCCCc1ccc(O)c(O)c1)O[C@H]1COC2C1OC[C@H]2OC(=O)CSCCCc1ccc(O)c(O)c1]
or a polymeric compound containing a repeating unit according to general Formula II 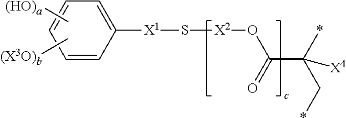
wherein, respectively independent of one another,
X¹ is a linear or branched alkylene radical having 2 to 18 carbon atoms,
X² is a linear or branched alkylene radical having 1 to 18 carbon atoms,
X³ is a linear or branched alkyl radical having 1 to 18 carbon atoms,
X⁴ is hydrogen or a linear or branched alkyl radical having 1 to 18 carbon atoms,
* is an attachment site of the repeating unit according to general Formula II,
A is a d-valent saturated or unsaturated group,
a is 2 to 5-b,
b is 0 to 3,
c is 0 or 1, and
d is 1 to 8.
